(19) United States
(12) Patent Application Publication
(10) Pub. No.: US 2025/0049946 A1
(43) Pub. Date: Feb. 13, 2025

(54) SULFONATO RESORCINARENE-COMPLEXED FLUOROQUINOLONES AND METHODS OF USE

(71) Applicant: University of Cincinnati, Cincinnati, OH (US)

(72) Inventors: Harshita Kumari, Mason, OH (US); Suchitra Dilipkumar Panigrahi, Seattle, WA (US); Ramana Reddy Mittapalli, Cincinnati, OH (US)

(21) Appl. No.: 18/800,769

(22) Filed: Aug. 12, 2024

Related U.S. Application Data
(60) Provisional application No. 63/531,954, filed on Aug. 10, 2023.

(51) Int. Cl. A61K 47/69

(52) U.S. Cl. CPC A61K 47/6949

(57) ABSTRACT

Supramolecular inclusion complexes comprising sulfonato methylresorcinarenes according to Formula I and a fluoroquinolone compound are provided. Also provided are pharmaceutical compositions comprising the supramolecular complexes and methods of using the supramolecular complexes to treat bacterial infections, kill or inhibit growth of bacteria, and disrupt bacterial biofilms.

### CROSS REFERENCE TO RELATED APPLICATION

This application claims priority to U.S. Provisional Application Ser. No. 63/531,954, filed Aug. 10, 2023, the entire contents of which are incorporated herein by reference.

### TECHNICAL FIELD

The present disclosure relates to the field of antibiotics and antibiotic resistance. Specifically, this disclosure relates to resorcinarene complexed fluoroquinolone antibiotics and their methods of use.

### BACKGROUND

Antibiotics have played a vital role in human and animal healthcare since the 1940s. During the “Golden Age” of antibiotic discovery (1940-1962), most of the antibiotic classes used today were discovered from natural microorganisms. However, this period of antibiotic discovery was followed by an “Innovation Gap,” due to the emergence of antibiotic-resistant bacterial strains. Efforts to modify existing antibiotic compounds faced obstacles, such as cost concerns and rapid development of resistance. This issue became evident with newly developed antibiotics, as seen in cases like methicillin-resistant Staphylococcus aureus (MRSA) shortly after the introduction of methicillin-based antibiotics. Overuse of antibiotics, driven by factors such as rising income and increased demand for animal protein, has exacerbated the problem. Despite steady growth in the antibiotic market, developing new antibiotics remains challenging, with low success rates and long development periods. Combating resistance involves neutralizing bacterial defense mechanisms through structural modifications or enhancing existing drug efficacy using carriers or combined therapeutics.

Nalidixic acid, considered the first quinolone drug (although structurally distinct from true quinolones) was introduced in 1962. Since then, successive generations of quinolones, including the popular fluoroquinolones, have been introduced for the treatment of a variety of infections. Fluoroquinolones inhibit bacterial enzymes required for DNA replication, making them effective against a broad spectrum of bacterial infections. However, their use is limited due to serious side effects and antibiotic resistance, posing a global health crisis. Efforts have been made to synthesize derivatives and gauge their efficacy against diverse microbial infections. New generations of fluoroquinolones have resulted in significant enhancements in their potency, range of action, and physical characteristics. The exploration of structure-activity relationships (SAR) has provided insights into the key elements responsible for fluoroquinolone effectiveness, such as the presence of a fluorine atom and the 1-alkyl, 1,4-dihydro-4-oxo-quinoline-3-carboxylic acid structure. It is further believed that the fluoro and piperazinyl groups play key roles in expanding the spectrum of activity and improving antipseudomonal properties. Investigations into chemical modifications at piperazinyl groups have sought to better regulate the pharmacokinetic properties and enhance the cell permeability of these promising antibacterial agents.

A need exists for improved antibiotic agents that avoid the adverse clinical reactions and antibiotic resistance associated with existing generations of fluoroquinolone drugs.

### SUMMARY

Provided herein are supramolecular complexes comprising a sulfonato resorcinarene macrocycle compound and a fluoroquinolone compound. The disclosed supramolecular complexes have broad-spectrum antibacterial efficacy, including efficacy against bacteria otherwise resistant to fluoroquinolone treatment.

In one embodiment, a supramolecular complex comprising a macrocycle compound according to Formula I is provided:

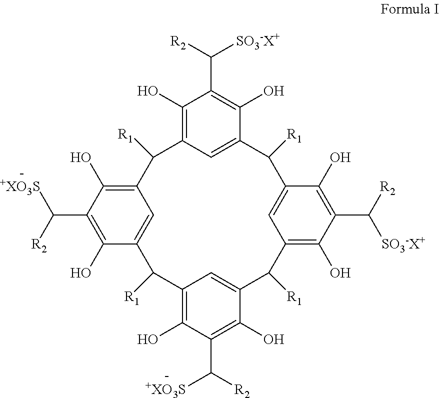

wherein: R₁ is selected from C₁-C₁₁ alkyl, C₁-C₁₁ alkoxy, and phenyl; and R₂ is selected from H, C₁-C₁₁ alkyl, C₁-C₁₁ alkoxy, phenyl, and (CH₂)ₙSO₃⁻X⁺, wherein n is an integer from 1 to 17 and X⁺ is a cation; and a fluoroquinolone compound.

In another embodiment, a pharmaceutical composition is provided, comprising a supramolecular complex comprising a macrocycle according to Formula I and a fluoroquinolone compound; and a pharmaceutically acceptable carrier.

In another embodiment, a method of treating a bacterial infection in a subject in need thereof is provided, the method comprising administering to the subject an effective amount of a supramolecular complex comprising a macrocycle according to Formula I and a fluoroquinolone compound.

In another embodiment, a method of killing or inhibiting the growth of bacteria is provided, the method comprising contacting the bacteria with an effective amount of a supramolecular complex comprising a macrocycle according to Formula I and a fluoroquinolone compound.

In another embodiment, a method of disrupting a bacterial biofilm is provided, the method comprising contacting the bacterial biofilm with an effective amount of a supramolecular complex comprising a macrocycle according to Formula I and a fluoroquinolone compound.

In another embodiment, supramolecular complex is provided, comprising: sulfonato-C-methylresorcin[4]arene (SRsC1) and a fluoroquinolone compound, or a salt thereof.

These and other features, aspects, and advantages will become better understood with reference to the following description and the appended claims.

Additional features and advantages of the embodiments described herein will be set forth in the detailed description that follows, and in part will be readily apparent to those skilled in the art from that description or recognized by practicing the embodiments described herein, including the detailed description that follows, the claims, as well as the appended drawings.

### BRIEF DESCRIPTION OF THE DRAWINGS

FIG. 1 depicts the chemical structure of sulfonato-C-methylresorcin[4]arene (SRsC1), according to embodiments of the present disclosure.

FIG. 2 depicts the chemical structure of β-cyclodextrin (βCD).

FIG. 3 depicts the chemical structures of levofloxacin, or LEVO (top panel), norfloxacin, or NOR (middle panel), and ciprofloxacin, or CIPRO (bottom panel).

FIG. 4 is a Job's Plot of selected protons of FQ guests (LEVO with PCD, LEVO, CIPRO, and NOR) with SRsC1 in D₂O except (CIPRO and NOR in 5% NaOD in D₂O), at 298 K.

FIG. 5 is a table showing proton NMR chemical shifts of host (H) and guest (G) protons in free and complex form in D₂O (except CIPRO and NOR in 5% NaOD in D₂O), at 298 K.

FIG. 6 is a table showing association constants and stoichiometry of all complexes in D₂O (except CIPRO and NOR in 5% NaOD in D₂O), at 298 K.

FIG. 7 is a non-linear curve fitting for the complexation between guests (LEVO, CIPRO and NOR) and host (PCD and SRsC1), in D₂O (except CIPRO and NOR in 5% NaOD in D₂O) at 298 K.

FIG. 8 is a table showing 2D DOSY ¹H-NMR diffusion coefficients (D, m²/s) of host and guest protons in free and complex form in 5% NaOD in D₂O, at 298 K.

FIGS. 9A-9D depict ¹H-¹H NOE spectra of equimolar mixtures of SRsC1-LEVO (FIG. 9A), βCD-LEVO (ROSEY) (FIG. 9B), SRsC1-CIPRO (FIG. 9C), and SRsC1-NOR (FIG. 9D) at 0.05 M concentration of each molecule in 5% NaOD in D₂O run at 283.15 K. Interactions between two protons are represented as same pattern circles or oval.

FIGS. 10A-10D depict comparative FTIR spectra of individual components, equimolar physical mixture (PM) and freeze-dried (FD) complex of SRsC1-LEVO (FIG. 10A), PCD-LEVO (FIG. 10B), SRsC1-NOR (FIG. 10C), and SRsC1-CIPRO FIG. 10D).

FIGS. 11A-11D are graphs depicting the differences between COMhost and the COMguest. FIG. 11A depicts SRsC1-LEVO and PCD-LEVO when the LEVO is started within SRsC1/PCD. FIG. 11B depicts SRsC1-LEVO and PCD-LEVO when the LEVO is started above SRsC1/PCD. FIG. 11C depicts SRsC1-CIPRO and SRsC1-NOR when the CIPRO/NOR is started within SRsC1. FIG. 11D depicts SRsC1-CIPRO and SRsC1-NOR when the CIPRO/NOR is started above SRsC1.

FIG. 12A depicts the structural interaction of SRsC1-LEVO complex.

FIG. 12B depicts the structural interaction of SRsC1-CIPRO complex.

FIG. 12C is a table showing exemplary binding enthalpies of guest-host complexes.

FIGS. 13A-13C are graphs depicting the impact of FQ complexes on S. aureus (SA) and P. aeruginosa (PA) for CIPRO and SRsC1-CIPRO (FIG. 13A), NOR and SRsC1-NOR (FIG. 13B), and LEVO, SRsC1-LEVO, and BCD-LEVO (FIG. 13C).

FIGS. 14A-14D are graphs depicting the effect of FQ complexes against resistant strains (R) of P. aeruginosa for CIPRO, SRsC1-CIPRO (FIG. 14A); NOR, SRsC1-NOR (FIG. 14B); LEVO, SRsC1-LEVO (FIG. 14C); and PCD-LEVO (FIG. 14D). Values represent the mean±S.D. (standard deviation). Student's paired two-tailed t-test, * statistically significant value with p<0.05 for macrocycle-FQ complexes vs. FQs (control).

FIGS. 15A-15C are graphs depicting the effect of free and complexed FQs against sensitive (S) and resistant (R) strains of P. aeruginosa for CIPRO and SRsC1-CIPRO (FIG. 15A); NOR and SRsC1-NOR (FIG. 15B); LEVO, SRsC1-LEVO, and PCD-LEVO (FIG. 15C).

### DETAILED DESCRIPTION

The details of embodiments of the presently disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently disclosed subject matter. Unless defined otherwise, all technical and scientific terms used herein have the same meaning as commonly understood by one of ordinary skill in the art to which the presently disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to define the disclosed subject matter and perform the disclosed method(s).

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an” and “the” include plural references unless the content clearly dictates otherwise.

As used herein, the term “subject” generally refers to a living being (e.g., animal or human) capable of suffering from a bacterial infection. In a specific embodiment, the subject is a mammal, such as a human, rat, mouse, monkey, horse, cow, pig, dog, cat, guinea pig, etc. In a specific embodiment, the subject is a human subject, a rat, or a mouse. In a more specific embodiment, the subject is a human.

The terms “treat,” “treatment,” and “treating,” as used herein, refer to a method of alleviating or abrogating a disease, disorder, and/or symptoms thereof. In a specific embodiment, the disease or disorder is a bacterial infection, including a biofilm colonization. In another specific embodiment, the bacterial infection is caused by Gram-negative or Gram-positive bacteria. In another embodiment, the bacteria is sensitive or resistant to treatment by fluoroquinolone compounds in free form (i.e., fluoroquinolones administered alone, and not as a supramolecular complex as disclosed herein).

As used herein, the terms “administer” or “administration” may comprise administration routes such as enteral (e.g., oral, sublingual, buccal, or rectal), parenteral (e.g., intravenous, intramuscular, subcutaneous, intraarterial, intrathecal, etc.), intranasal, inhaled, vaginal, transdermal, topical (including ophthalmic administration via eye drops), etc., so long as the route of administration results in treatment of a bacterial infection or biofilm. In specific embodiments, the administration route is enteral, parenteral, topical, or ophthalmic.

“Effective amount,” as used herein, refers to an amount of an agent sufficient to achieve a desired biological effect. In methods of treating a subject, effective amounts will vary based on a subject's age, body weight, condition, and the like, and may be determined by one of skill in the art in view of the present disclosure. The supramolecular complexes and compositions of the present disclosure can be administered by either single or multiple dosages of an effective amount. In embodiments, the effective amount of an agent is an amount sufficient to kill bacteria, inhibit bacterial growth, and/or disrupt a biofilm.

A bacterial biofilm is a three-dimensional community of bacteria that adhere to each other, and often to a surface. Cells within a biofilm communicate via quorum sensing to regulate metabolic activity. Biofilms coordinate via signaling networks to protect the component cells from external stresses. Cells in a biofilm become embedded within an extracellular matrix that makes the biofilm more difficult to eradicate. Disruption of a biofilm results in the release of bacterial cells from the biofilm to a planktonic state, where cells are more vulnerable to eradication via antibiotics. In embodiments, “disruption” refers to the partial or complete removal of a biofilm or biofilm matrix, and/or damage to the integrity of a biofilm.

Calixarenes and closely related resorcinarenes are macrocyclic supramolecular hosts with highly versatile structures. These compounds possess a concave binding cavity that exhibits a strong affinity for a wide range of guest molecules, including cations (alkali and alkaline earth metals, transition metals, and ammonium ions), anions, and small organic compounds. Resorcinarenes, cyclic polyphenols formed through the condensation of resorcinol with different aldehydes in acidic solution, are remarkably adaptable, as they can incorporate distinct substituents in the electron-rich upper rim groups and lower rim alkyl chains. In addition, resorcinarenes bearing sulfonate groups have anionic properties, resulting in water solubility, and an electron-rich hydrophobic interior cavity that can interact with various guests through interactions such as hydrogen bonding and π-interactions. Because of their exceptional adaptability and high affinity for hydrogen bonding, resorcin[4]arenes are particularly suitable for host-guest chemistry.

Inclusion of sulfonato groups in the resorcinarene upper rim advantageously increases solubility of the macrocycles disclosed herein. Enhanced water solubility of macrocycles is beneficial for biological applications and improves the binding affinity with the guest molecule. It has now been found that adding sulfonato groups to the resorcinarene macrocycle introduces additional sites for non-covalent interactions with the guest molecule. This improved binding affinity can increase the overall enthalpy of the supramolecular complex, thereby enhancing the solubility, dissolution, and bioavailability profile for drug release. Described herein are supramolecular sulfonato resorcinarene complexes comprising a guest fluoroquinolone, pharmaceutical compositions comprising the same, and their use in antibacterial applications.

### Sulfonato Resorcinarene Complexes

In embodiments, a supramolecular complex is provided, comprising a macrocycle compound according to Formula I:

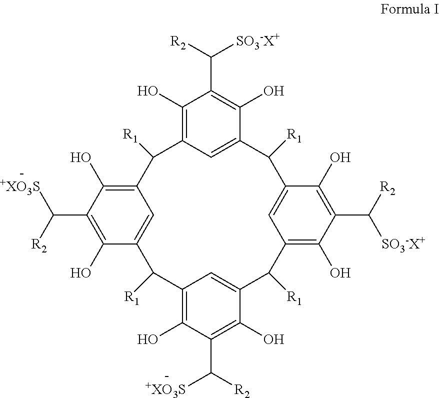

wherein:
R₁ is selected from C₁-C₂₀ alkyl, C₁-C₂₀ alkoxyl, and aryl; and
R₂ is selected from H, C₁-C₂₀ alkyl, C₁-C₂₀ alkoxyl, aryl, and (CH₂)ₙSO₃⁻X⁺, wherein n is an integer from 1 to 17 and X⁺ is a cation; and
a fluoroquinolone compound.

In specific embodiments, R₁ is selected from C₁-C₈ alkyl, C₁-C₈ alkoxyl, and phenyl. In more specific embodiments, R₁ is selected from C₁-C₄ alkyl, C₁-C₄ alkoxyl, and phenyl. In more specific embodiments, R₁ is selected from methyl, ethyl, methoxyl, ethoxyl, and phenyl. In a very specific embodiment, R₁ is methyl. In embodiments, each R₁ moiety has the same identity.

In specific embodiments, R₂ is selected from H, C₁-C₈ alkyl, C₁-C₈ alkoxyl, phenyl, and (CH₂)ₙSO₃⁻X⁺, wherein n is an integer from 1 to 17 (i.e., n=1, 2, 3, 4, 5, 6, 7, 8, 9, 10, 11, 12, 13, 14, 15, 16, 17, or any range defined by any two integers within the range) and X⁺ is a cation. For example, in embodiments, R₂ is (CH₂)ₙSO₃⁻X⁺, wherein n is an integer from 1 to 2, from 1 to 3, from 1 to 4, from 1 to 5, from 1 to 6, from 1 to 7, from 1 to 8, from 1 to 9, from 1 to 10, from 1 to 11, from 1 to 12, from 1 to 13, from 1 to 14, from 1 to 15, from 1 to 16, from 1 to 17, from 2 to 3, from 2 to 4, from 2 to 5, and so forth. In more specific embodiments, R₂ is selected from H, C₁-C₄ alkyl, C₁-C₄ alkoxyl, phenyl, and (CH₂)ₙSO₃⁻X⁺, wherein n is an integer from 1 to 8. In a very specific embodiment, R₂ is selected from H or methyl. In embodiments, each R₂ moiety has the same identity.

In embodiments, X⁺ is a cation associated with the SO₃⁻ sulfonato group of the resorcinarene upper-rim. The skilled artisan will appreciate that the identity of the cation will vary, depending on the salt form of the sulfonato methylresorcinarene selected. In embodiments, the cation is selected from the group consisting of sodium (Na⁺), potassium, (K⁺), ammonium (NH₄+), pyridinium (C₅H₅NH⁺), and the like. In a specific embodiment, the cation is Na⁺.

As used herein, “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, alkyl refers, in particular, to C₁₋₈ straight-chain alkyls. In other embodiments, alkyl refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to substituted or unsubstituted alkyl, alkoxyl, halo, hydroxyl (—OH), cyano (—CN), carboxyl, carboxyl ester, aryloxyl, alkylthio, arylthio, aralkyloxyl, aralkylthio, alkoxycarbonyl, oxo, arylamino, acyl, and cycloalkyl. There can be optionally inserted along the alkyl chain one or more heteroatoms, such as oxygen, sulfur or substituted or unsubstituted nitrogen atoms, wherein the nitrogen substituent is hydrogen, lower alkyl (also referred to herein as “alkylaminoalkyl”), or aryl. In a specific embodiment, alkyl substitutions are selected from the group consisting of alkoxyl, halo, hydroxyl (—OH), cyano (—CN), carboxyl, carboxyl ester, substituted or unsubstituted alkyl, and combinations thereof.

Thus, as used herein, the term “substituted alkyl” includes alkyl groups, as defined herein, in which one or more atoms or functional groups of the alkyl group are replaced with another atom or functional group, including for example, alkoxyl, halo, hydroxyl (—OH), cyano (—CN), carboxyl, carboxyl ester, and substituted or unsubstituted alkyl.

“Alkoxyl” refers to an alkyl-O— group wherein alkyl is as previously described. The term “alkoxyl” as used herein can refer to, for example, methoxyl, ethoxyl, propoxyl, isopropoxyl, butoxyl, t-butoxyl, and pentoxyl. The term “oxyalkyl” can be used interchangably with “alkoxyl.”

The term “aryl” refers to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings. In a very specific embodiment, the aryl moiety is phenyl.

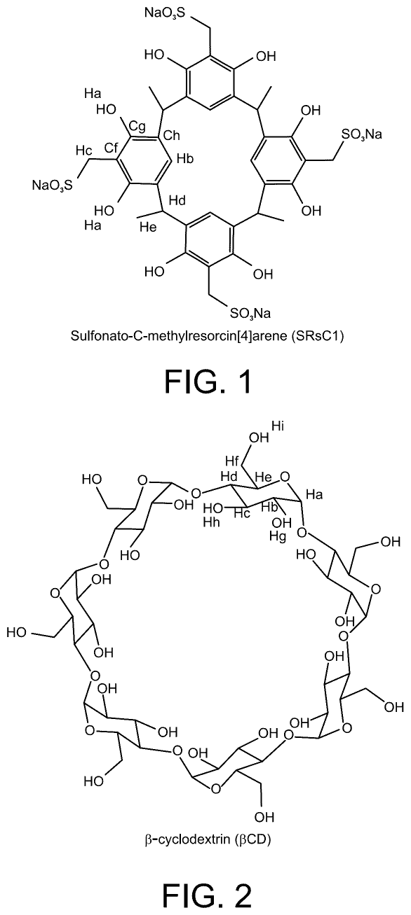

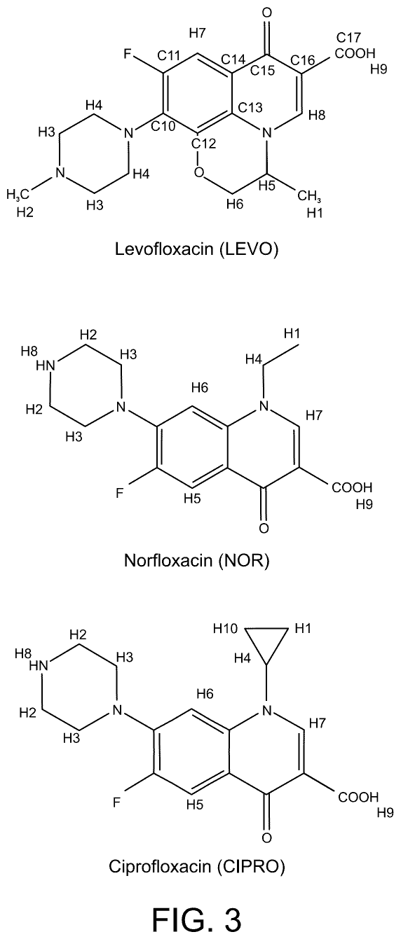

In specific embodiments, the macrocycle compound of the disclosed supramolecular complexes is sulfonato-C-methylresorcin[4]arene (SRsC1). In embodiments, the structure of SRsC1 is set forth in FIG. 1.

Quinolone antibiotics are broad-spectrum bacteriocidals that share a bicyclic core structure. Fluoroquinolones contain a fluorine atom in their chemical structure and are effective against both Gram-negative and Gram-positive bacterial. In embodiments, the fluoroquinolone compound of the disclosed supramolecular complexes is selected from the group consisting of ciprofloxacin, levofloxacin, norfloxacin, delafloxacin, gemifloxacin, moxifloxacin, sparfloxacin, and ofloxacin. In specific embodiments, the fluoroquinolone is selected from the group consisting of ciprofloxacin, levofloxacin, and norfloxacin.

In embodiments, the supramolecular complex of the present disclosure has a binding stoichiometry of macrocycle to fluoroquinolone of about 1:1.

### Synthesis of Sulfonato Resorcinarenes

In embodiments, sulfonato resorcinarenes according to the present disclosure are synthesized according to Scheme 1:

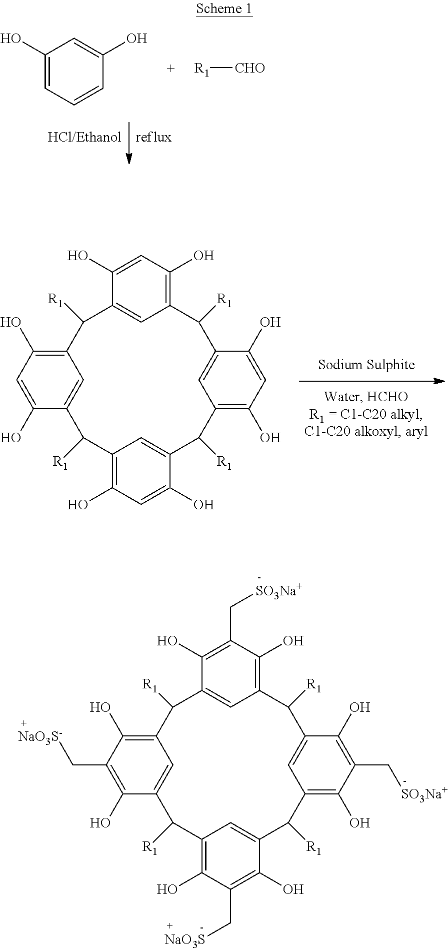

Derivatization of RsC1 with sulfonato groups is achieved by treating cyclized RsC1 with aldehyde and sodium sulfite. The hydrophilicity and lipophilicity can be adjusted by using different aldehydes during the cyclization process. Additionally, the sulfonato group can be modified to optimize host-guest complexation (for example, via Schemes 2 and 3, described herein below), allowing for fine-tuning of the hydrophilicity-lipophilicity index to target selective receptor sites.

In embodiments, branched sulfonato resorcinarenes are synthesized according to Scheme 2:

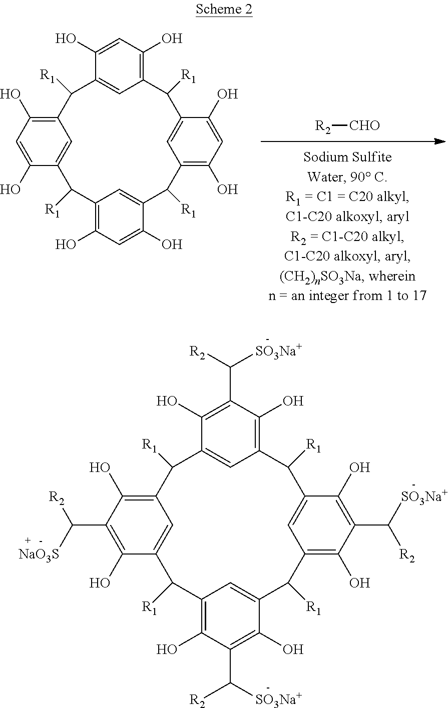

Briefly, calix[4]resorcinarene (RsC1) (0.01 mol), a solution of 37% formaldehyde or aliphatic/aromatic aldehyde (0.05 mol) and sodium sulfite (0.05 mol) in water (30 ml) is stirred and reflux for 4 h. Dilute hydrochloric acid is added after cooling until pH 7, then acetonitrile (50 ml) is added to precipitate the product. The solid is filtered, washed with acetone/acetonitrile (1:1, 20 ml) and dried under high vacuum for overnight. Compound SRsC1 is a light brown powder with a yield of 48%.

In embodiments, alkyl branched sulfonato resorcinarenes are synthesized according to Scheme 3, wherein n=an integer from 1 to 17:

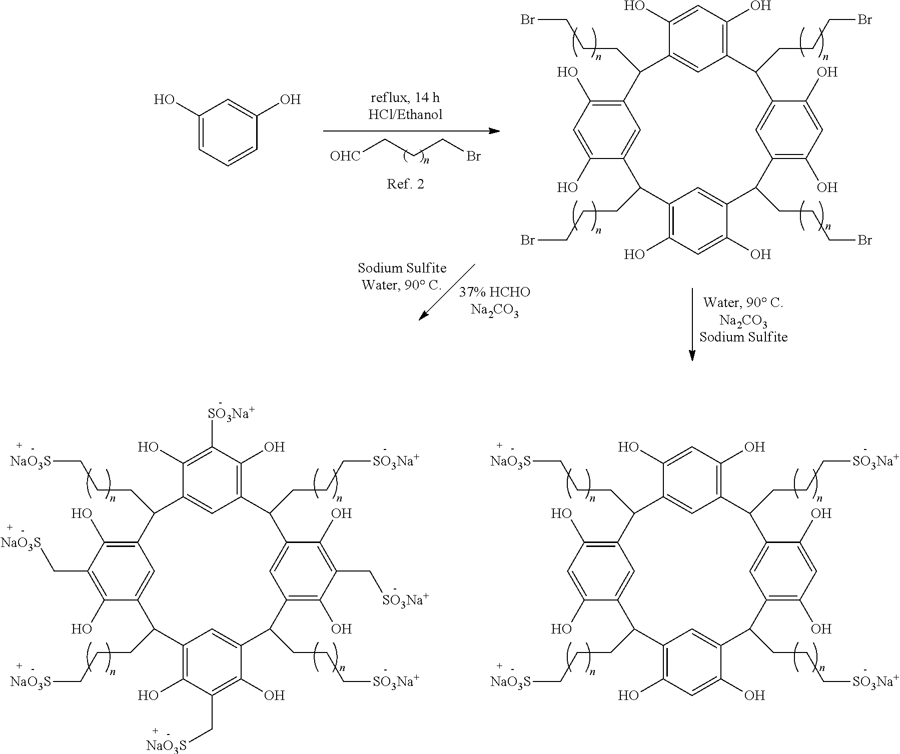

Briefly, bromo resorcinarenes (BrRsC₃) (0.01 mol) and 37% HCHO (0.05 mol)/sodium sulfite (0.05 mol), Na₂CO₃ (0.05 mol) in water (30 ml) is refluxed for 4 h. Dilute hydrochloric acid is added until pH 7, then acetonitrile (50 ml) is added to precipitate the product. The solid is filtered and washed with acetone/acetonitrile (1:1, 20 ml) and dried under high vacuum overnight to yield an alkyl branched sulfonato resorcinarene (SRsC₃) product.

### Synthesis of Supramolecular Complexes

Macrocylic sulfonato resorcinarenes are complexed with fluoroquinolone compounds to provide the supramolecular complexes disclosed herein. Briefly, sulfonato resorcinarene and fluoroquinolone are mixed in 1:1 mole ratio in water at room temperature for 24 h. The solid complex is obtained by freeze-drying the 24-hour mixed solution after passing it through a 0.45 mm membrane filter.

### Pharmaceutical Compositions

The supramolecular complexes disclosed herein, comprising a macrocycle compound according to Formula I and a fluoroquinolone, may be referred to as “active agents.” Pharmaceutical formulations comprising the aforementioned active agents also are provided herein. These pharmaceutical formulations comprise active agents as described herein, in a pharmaceutically acceptable carrier. Pharmaceutical formulations can be prepared for oral, intravenous, or topical administration as discussed herein. Also, the presently disclosed subject matter provides such active agents that have been lyophilized and that can be reconstituted to form pharmaceutically acceptable formulations (including formulations pharmaceutically acceptable in humans) for administration.

In embodiments, the supramolecular complexes disclosed herein may be used or administered in their free or salt form. In embodiments, administration of the disclosed supramolecular complexes in salt form may enhance hygroscopicity, solubility, dissolution, physical stability, chemical stability, and/or processability. Selection of a suitable salt form is within the purview of the ordinary skilled artisan. See Byrn, et al., Solid State Properties of Pharmaceutical Materials, Chapter 4, Pharmaceutical Salts (2017); Remington: The Science and Practice of Pharmacy (23rd ed., Adeboye Adejare, ed., 2020).

In embodiments, the pharmaceutically acceptable salt form is selected from the group consisting of hydrochloride, sodium, sulfate, acetate, phosphate, diphosphate, chloride, potassium, malate, calcium, citrate, mesylate, nitrate, tartrate, aluminum, gluconate, and the like. In embodiments, the negatively charged SO₃ groups of the Formula I compound are associated with a positively charged cation. In specific embodiments, the cation may be a sodium ion (Na⁺), a potassium ion (K⁺), or another suitable cation, based on the salt form selected. In specific embodiments, the cation is selected from the group consisting of Na⁺, K⁺, NH₄+, and C₅H₅NH⁺.

The therapeutically effective dosage of any specific active agent, the use of which is within the scope of embodiments described herein, will vary somewhat from agent to agent, and subject to subject, and will depend upon the condition of the subject and the route of delivery. As a general proposition, a unit dosage from about 10 mg to about 1000 mg will have therapeutic efficacy, with all weights being calculated based upon the weight of the active complex, including the cases where a salt is employed. The duration of the treatment is usually 1-2 unit doses per day for a period of 3 to 14 days, or until the condition is controlled.

As will be understood by those of skill in this art, the specific dose level for any particular subject will depend on a variety of factors, including the activity of the agent employed; the age, body weight, general health, and sex of the subject being treated; the time and route of administration; the rate of excretion; and the like.

In embodiments, pharmaceutical compositions may be formulated for enteral (e.g., oral, sublingual, buccal, or rectal), parenteral (e.g., intravenous, intramuscular, subcutaneous, intraarterial, intrathecal, etc.), intranasal, inhaled, vaginal, transdermal, topical (including ophthalmic administration via eye drops), administration.

In a specific embodiment, the pharmaceutical composition is formulated for enteral administration, and more specifically for oral administration as a liquid or solid dosage form.

In a specific embodiment, the pharmaceutical composition is formulated for parenteral administration, and more specifically for intravenous administration via injection or infusion. For parenteral administration, suitable compositions include aqueous and non-aqueous sterile suspensions for intravenous administration. The compositions may be presented in unit dose or multi-dose containers, for example, sealed vials and ampoules.

In another specific embodiment, the pharmaceutical composition is formulated for topical or ophthalmic administration. For example, the pharmaceutical composition may be formulated for administration as a cream, ointment, lotion, solution, suspension, gel, foam, spray, eye drop, or the like.

The compositions may be prepared by any methods well known in the art of pharmacy, for example, using methods such as those described in Remington: The Science and Practice of Pharmacy (23rd ed., Adeboye Adejare, ed., 2020, see Section 7: Pharmaceutical Materials and Devices/Industrial Pharmacy). Suitable pharmaceutical carriers are well known in the art. See, for example, Handbook of Pharmaceutical Excipients, Sixth Edition, edited by Raymond C. Rowe (2009). The skilled artisan will appreciate that certain carriers may be more desirable or suitable for certain modes of administration of an active ingredient. It is within the purview of the skilled artisan to select the appropriate carrier(s) for a given pharmaceutical composition and route of administration.

### Methods of Use

Provided herein are methods of treating bacterial infections in a subject in need thereof, comprising administering to the subject an effective amount of a supramolecular complex comprising: a macrocycle compound according to Formula I and a fluoroquinolone compound, as described herein.

In a specific embodiment, the macrocycle compound is sulfonato-C-methylresorcin[4]arene (SRsC1), or a pharmaceutically acceptable salt thereof.

In embodiments, the fluoroquinolone compound is selected from the group consisting of ciprofloxacin, levofloxacin, norfloxacin, delafloxacin, gemifloxacin, moxifloxacin, sparfloxacin, and ofloxacin. In a specific embodiment, the fluoroquinolone compound is ciprofloxacin, levofloxacin, and/or norfloxacin.

Various bacterial infections are suitable for treatment according to the disclosed methods. In embodiments, the bacterial infection is selected from the group consisting of a respiratory infection, a urinary tract infection, a joint infection, a bone infection, an intraabdominal infection, a soft tissue infection, a skin infection, an eye infection, a gastrointestinal infection, septicemia, typhoid fever, and anthrax.

In a specific embodiment, the bacterial infection is caused by a bacterium selected from the group consisting of Acinetobacter spp., Actinomycetes spp., Bacillus spp., Bacteroides spp., Bordeiella spp., Borrelia spp., Branhamela spp, Brucella spp., Burkholderia spp., Cedecea spp., Chlamydia spp., Citrobacer spp., Clostridium spp., Coxiella spp., Edwardsiella spp., Enterobacter spp., Enterobacteriaceae spp., Enterococcus spp., Escherichia spp., Erysipelothrix spp., Ewingella spp., Francisella spp., Fusospirocheia spp., Gardnerella spp., Haemophilus spp., Hafnia spp., Klebsiella spp., Kluyvera spp., Legionella spp., Listeria spp. Micrococcus spp., Morganella spp., Mycobacteria spp., Mycoplasma spp., Neisseria spp., Nocardia spp, Pasteurella spp., Peptostreptaoccus spp., Proteus spp., Providencia spp., Pseadomonas spp., Rahnella spp., Rickettsia spp., Salmonella spp., Serratia spp., Shigella spp., Spirillum spp., Spirochaeta spp., Staphylococcus spp. Streptobacillus spp. Streptococcus spp., Streptomyces spp., Tatumella spp., Treponema spp., Trichomonas spp., Ureaplasma spp., Vaginalis spp., Vibrio spp., Xanthomonas spp., and Yersinia spp.

In a more specific embodiment, the bacterial infection is caused by Staphylococcus aureus or Pseudomonas aeruginosa.

In embodiments, the bacterial infection is caused by Gram-positive and/or Gram-negative bacteria.

In embodiments, the bacterial infection is caused by bacteria that are sensitive or resistant to fluoroquinolone compounds, such as the fluoroquinolone compound included in the administered supramolecular complex.

In embodiments, risk of developing antibiotic resistance to the fluoroquinolone compound included in the supramolecular complex is reduced when the fluoroquinolone compound is administered as part of the disclosed supramolecular complexes.

Also provided herein are methods of killing or inhibiting the growth of bacteria, the method comprising contacting the bacteria with an effective amount of a supramolecular complex as disclosed herein, that is, a supramolecular complex comprising a macrocycle according to Formula I and a fluoroquinolone compound, or a pharmaceutically acceptable salt thereof. In embodiments, the method is suitable for in vitro or in vivo use.

In a specific embodiment, the macrocycle compound is sulfonato-C-methylresorcin[4]arene (SRsC1), or a pharmaceutically acceptable salt thereof.

In embodiments, the fluoroquinolone compound is selected from the group consisting of ciprofloxacin, levofloxacin, norfloxacin, delafloxacin, gemifloxacin, moxifloxacin, sparfloxacin, and ofloxacin. In a specific embodiment, the fluoroquinolone compound is ciprofloxacin, levofloxacin, and/or norfloxacin.

In a specific embodiment, bacteria are selected from the group consisting of Acinetobacter spp., Actinomycetes spp., Bacillus spp. Bacteroides spp., Bordetella spp., Borrelia spp., Branhamnella spp., Brucella spp., Burkholderia spp., Cedecea spp., Chlamydia spp., Citrobacter spp., Clostridium spp., Coxiella spp., Edwardsiella spp., Enterobacter spp, Enterobacteriaceae spp., Enterococcus spp., Erwinia spp., Escherichia spp., Erysipelothrix spp., Ewingella spp., Francisella spp., Fusospirocheta spp., Gardenerella spp., Haemophilus spp., Hafnia spp., Klebsiella spp., Kluyvera spp., Legionella spp., Listeria spp., Micrococcus spp., Morganella spp., Mycobacteria spp., Mycoplasma spp., Neisseria spp., Nocardia spp, Pasteurella spp., Peptostreptococcus spp., Proteus spp., Providencia spp., Pseudomonas spp., Rahnella spp., Rickettsia spp., Salmonella spp., Serratia spp., Shigella spp., Spirillum spp., Spirochaeta spp., Staphylococcus spp., Streptobacillus spp., Streptococcus spp., Streptomyces spp., Tatumella spp., Treponema spp., Trichomonas spp., Ureaplasma spp., Vaginalis spp., Vibrio spp, Xanthomonas spp., and Yersinia spp.

In a more specific embodiment, the bacteria are selected from Staphylococcus aureus or Pseudomonas aeruginosa.

In embodiments, the bacteria are Gram-positive or Gran-negative bacteria.

In embodiments, the bacteria are sensitive or resistant to fluoroquinolone compounds, such as the fluoroquinolone compound included in the administered supramolecular complex.

In embodiments, risk of developing antibiotic resistance to the fluoroquinolone compound included in the supramolecular complex is reduced when the fluoroquinolone compound is administered as part of a supramolecular complex as disclosed herein.

Also provided herein is a method of disrupting a bacterial biofilm, the method comprising contacting the bacterial biofilm with a supramolecular complex of the present disclosure. In embodiment, the supramolecular complex comprises a macrocycle compound according to Formula I and a fluoroquinolone compound, or a salt form thereof.

In a specific embodiment, the macrocycle compound is sulfonato-C-methylresorcin[4]arene (SRsC1) or a salt form thereof. In a specific embodiment, the fluoroquinolone compound is selected from the group consisting of ciprofloxacin, levofloxacin, norfloxacin, delafloxacin, gemifloxacin, moxifloxacin, sparfloxacin, and ofloxacin.

### EXAMPLES

The following examples are given by way of illustration are not intended to limit the scope of the disclosure.

### Example 1. Materials and Methods

### Sulfonato-C-methylresorcin[4]arene (SRsC1) and FQ Complexes

Sulfonato-C-methylresorcin[4]arene (SRsC1) was synthesized as described herein (see also Kazakova, et al., The Complexation Properties of the Water-Soluble Tetrasulfonatomethylcalix[4]resorcinarene toward α-Aminoacids, Journal of Inclusion Phenomena and Macrocyclic Chemistry, 43(1), 65-69 (2002)). All antibiotics, levofloxacin (LEVO), norfloxacin (NOR), ciprofloxacin (CIPRO) and β-cyclodextrin (βCD) were purchased from Sigma-Aldrich (USA). Deuterated water (D₂O) and sodium deuteroxide solution (NaOD) were purchased from Sigma-Aldrich (USA).

Sulfonato-C-methylresorcin[4]arene (SRsC1) host and each FQ guest, levofloxacin (LEVO), norfloxacin (NOR), ciprofloxacin (CIPRO) were mixed in 1:1 mole ratio in water at room temperature for 24 h. The solid complex was obtained by freeze-drying the 24-hour mixed solution after passing it through a 0.45 mm Millipore membrane filter. The freeze-drying process was carried out in a freeze dryer (ScanVac CoolSafe freeze dryer, Labogene, Denmark) for a duration of 24 hours. The physical mixture (PM) was prepared by blending host and FQ in 1:1 mole ratio. Likewise, β-cyclodextrin (βCD) and LEVO complex were prepared.

### Determination of Complex Stoichiometry and Association (Binding) Constant Via 1H-NMR Titrations

Titrations were carried out for all FQ guests (LEVO, CIPRO, and NOR) with macrocyclic host (SRsC1 and βCD). A stock solution containing 10 mM of each guest (LEVO, CIPRO, and NOR) and host (SRsC1 and βCD) was prepared. The solutions were mixed in different ratios (host:guest, 1:0, 1:1, 1:1.5, 1:2, 1:3, 1:4, 1:6, 1:9 and vice versa) and filled with the appropriate background media (D₂O) to a total volume of 700 μL and the NMR spectra were recorded after 24 h. The observed chemical shift (δ_{obs}) of a given nucleus can be expressed using the following formula:

$${δ}_{obs} = {δ}_{G} + Δ δ \frac{{[ G ]}_{T} + {[ H ]}_{T} + \frac{1}{K} - \sqrt{{( {[ G ]}_{T} + {[ H ]}_{T} + \frac{1}{K} )}^{2} - {4 [ G ]}_{T} + {[ H ]}_{T}}}{{2 [ G ]}_{T}} \qquad Equation ( 1 )$$

where Δδ=δ_{G-H}−δ_{G}.

The total concentration of mixture was constant during the titration with varying ratio of host and guest molecule. Binding constant (K) also known as association constant (Kₐ) of the inclusion complexes were calculated by non-linear parameter fitting of quadratic binding equation Eq. (1) to the δ_{obs} versus total concentration of host [H]_{T} datasets using solver program by known methods (Szabó, et al., Equilibrium, structural and antibacterial characterization of moxifloxacin-β-cyclodextrin complex, Journal of Molecular Structure, 1166, 228-236 (2018)).

### 2D ₁H-NMR Experiments

Diffusion-ordered spectroscopy (DOSY): DOSY spectra were collected with a stimulated echo pulse sequence with two spoil gradients. Smoothed rectangular pulses ranging from 0.66 ms to 1.84 ms were used ranging from approximately 50 to 1.0 G/cm in 16 increments with a diffusion time of 60 ms. Diffusion coefficients were calculated by fitting peak height as a function of gradient strength using the “dosy2d” program in Topspin 4.1. All NMR datasets were collected with a Bruker AVANCE NEO 400 MHz spectrometer equipped with a 5 mm broadband SmartProbe (Massachusetts, USA) associated with each peak. This method was used to separate peaks corresponding to different compounds in mixtures (SRsC1-LEVO, βCD-LEVO, SRsC1-CIPRO, and SRsC1-NOR), as well as to probe multimerization or intermolecular binding.

NOESY/ROESY: The molecular geometry of the complex was investigated by two-dimensional phase-sensitive rotating frame nuclear Overhauser effect spectroscopy (2D ROESY or NOESY). The structures of the inclusion complexes were determined applying a spinlock of 3 kHz for a mixing time of 0.5 ms. The samples used in the 2D NOESY/ROESY experiments consisted of equimolar amounts of ground FQs and macrocycle product in 10% NaOD in D₂O, with a concentration of 50 mM.

### FTIR Spectroscopic Measurements

IR spectra were recorded in the range of 4000-400 cm⁻¹ by means of a Nicolet 6700 FTIR spectrometer and Software version OMNIC 7.4.

### Computational Studies: Molecular Dynamics (MD) Simulations

The guest-host combinations SRsC1-LEVO, βCD-LEVO, SRsC1-CIPRO, and SRsC1-NOR were investigated in the presence of H₂O. Before solvating the system, host-guest systems were constructed by placing the guest in different orientations around the host. The guest orientations include encapsulation, where the piperazine group or the carboxylic group oriented within the host cavity through both the upper- and lower-rims, placing the guest to the side of the host, placing the guest outside of the upper- or lower-rim, and placing the guest ˜10 Å from the upper- or lower-rim. The isolated guest-host system was minimized, solvated with a 10 Å truncated octahedron with four randomly placed Na⁺ ions, and then minimized. The solvated-system was heated from 0-298.15 K over 60 ps. The system was equilibrated using a canonical ensemble (NVT) over 30 ps before running an NPT simulation over 50 ps. Further NPT production runs were carried out for 20 ns to monitor the behavior of the guest-host system. All simulations were carried out with the AmberTools suite of programs visualized with Chimera, and analysis was performed with CPPTraj.

To further probe the host-guest interaction and determine the binding enthalpy (ΔH), simulations were carried out for all the solvated host-guest combinations (with piperazine oriented within the host), solvated hosts (SRsC1 and βCD), solvated guests (LEVO, CIPRO, and NOR) and solvent. All these systems contained 1500 H₂O molecules and 4 Na⁺ ions. The same protocol for minimization, equilibration, and production runs was implemented as indicated above. The binding of the guest by the host (Equation (2)) was of interest so ΔH was calculated by using Equation (3) where the <E> for each system is the average potential energy; this approach has been implemented in other studies investigating the ΔH of host-guest systems. To determine ΔH, the potential energy is sampled every 0.1 ps and average is found for the 20 ns production run except for the first 50 ps, which is not included and is being used for further equilibration time. The uncertainty in the binding affinity was determined by using blocking analysis; for this study, a block size of 100 ps was used. The uncertainty for the binding affinity was determined by taking the square root of the square of the absolute binding affinity as found in Equation (4).

$$Host + Guest \to Host ‐ Guest \qquad Equation ( 2 )$$

$$Δ H = 〈 E 〉 host ‐ guest + 〈 E 〉 solvent - 〈 E 〉 host - 〈 E 〉 guest \qquad Equation ( 3 )$$

$$σ 2 Total = σ 2 host ‐ guest + σ 2 solvent + σ 2 host + σ 2 guest \qquad Equation ( 4 )$$

### Antibacterial Activity

The in vitro antibacterial activity was tested using a Gram-positive bacterial pathogen, S. aureus, and sensitive and resistant strains of a Gram-negative bacterial pathogen, P. aeruginosa using a high-throughput format in a 96-well microtiter plate, as described previously. Briefly, an inoculum of the test organism was grown to mid-log phase in a Luria broth (LB) medium containing 10 g/L tryptone, 5 g/L yeast extract, and 10 g/L sodium chloride. A 50 μL aliquot of the 100-fold diluted bacterial inoculum was placed in each test well. Individual test concentrations (in triplicate wells in a 96-well plate) for a given molecule were achieved by serial dilution with the LB medium except NOR and CIPRO. Because of poor solubility, NOR and CIPRO solubilizes in 10% v/v DMSO (inert solvent) in LB media. With S. aureus and P. aeruginosa, the final concentration range for test compounds were 128-0.03 μg/mL and 36-0.06 μg/mL. The plates were incubated in a 37° C. incubator for 24 h. Microbial growth in the test wells was monitored by reading the absorbance at 600 nm using a Biotek Epoch2 microplate reader. The MIC90 was defined as the minimum inhibitory concentration of the drug at which ≥90% growth of the test organism was inhibited. All experiments were performed in triplicate. For each measurement, three samples in each replicate were analyzed and data were recorded as mean±standard deviation (SD). A paired two-tailed t-test was employed for mean comparison, with significance determined at p<0.05. The dcata analysis was conducted using the Excel program.

### Determination of Partition Coefficient

The experimental partition coefficient (Ko/w) between n-octanol and phosphate buffer was determined by a slight modification of known methods. Briefly, 80 μL of a sample stock solution (0.3 mg/mL) was diluted with 1.9 mL of appropriate phosphate buffer solution (pH 7.4) and mixed with 2 mL of octanol (the organic and aqueous phase was mutually saturated), the vials were protected from light by wrapping in aluminum foil. For Ko/w of complexed FQs with hosts, SRsC1 and BCD, 10 times higher stock solutions (3 mg/mL) of host were added in aqueous phase containing guest FQs. The two phases were vortexed for 1 min and agitated at 120 rpm for 24 h in a shaker at 25±0.1° C. After equilibration, the aqueous phase was removed with a pasteur pipette and both phases were assayed spectrometrically to determine drug concentration. The partition coefficient was calculated as the ratio between molar concentration in n-octanol and aqueous phase. The total concentration in both phases was measured by spectrophotometry and the experimental partition coefficients (Ko/w) were determined from Equation (5).

$$K o / w = Co / Cw \times Vw / Vo \qquad Equation ( 5 )$$

where Co and Cw represent the solute concentration in the octanol and aqueous phase after distribution, respectively; Vw represents the volume of the aqueous phase and Vo the volume of the organic phase.

All partition coefficient determinations were made in triplicate. Fluorescence spectrophotometer (Varioskan Lux with the SkanIt 4.1 application software (USA)) with micro plate were used throughout this study for all measurements.

### Example 2. Stoichiometry of Complex and Association Constant

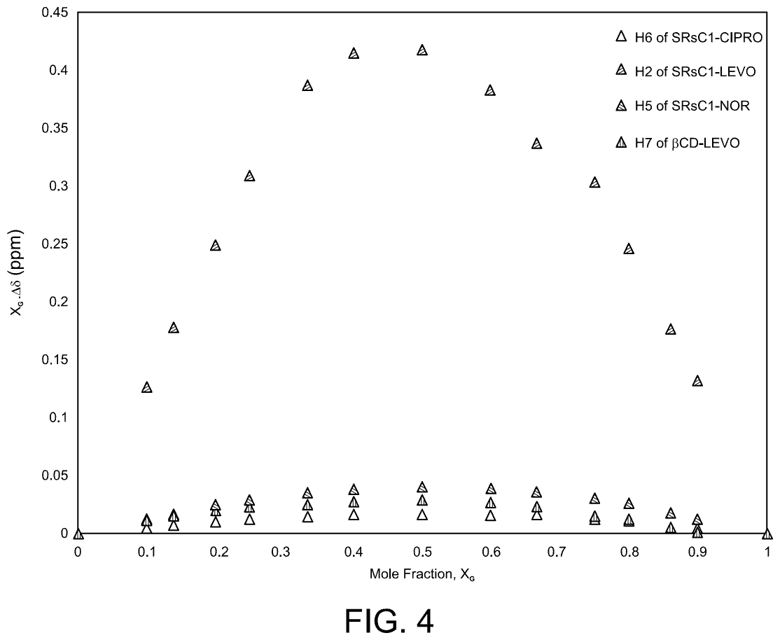

The continuous variation method (Job's method) was used to determine the FQ guests' inclusion complex stoichiometry, which is useful for characterization of host-guest interactions. During the Job's plot titration, the ¹H-NMR chemical shifts (δ) were measured at different guest (G)/host (H) concentration (cG/cH) ratios while total concentration (cG+cH) was kept constant. The calculated factors (Δδ *X_{G}), i.e., difference in chemical shift of protons between host-guest mix (δ_{G-H}) and guest (δ_{G}) multiplied with molar ratio of guest (X_{G}) were plotted against X_{G}. All titration curves showed a maximum at X_{G}=0.5 for all the tracked nuclei, suggesting a 1:1 complex stoichiometry (see FIG. 4).

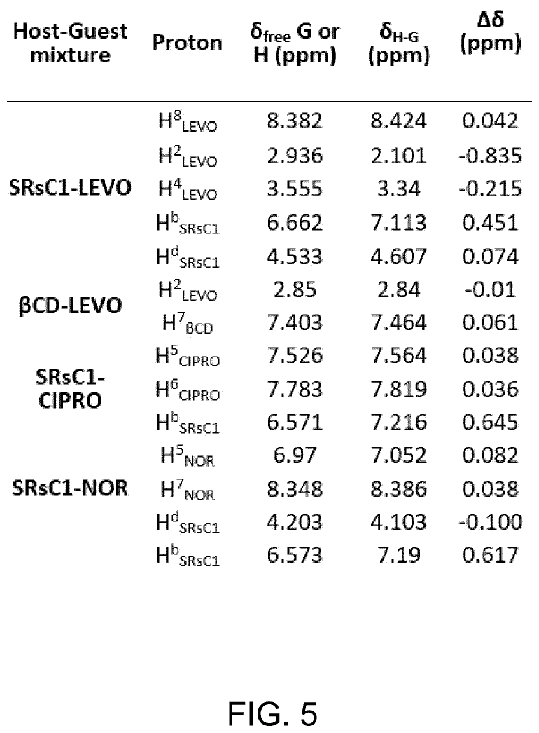

The chemical shifts (δ) for selected protons of the FQ guests and macrocycle hosts in the free and complexed states are summarized in the table of FIG. 5 at equimolar host/FQ guest ratios.

A positive sign for Δδ indicates a downfield shift, whereas a negative sign indicates an upfield shift. No new peaks appeared in the ¹H-NMR spectra of the inclusion complexes (FIG. 4), indicating that the inclusion of the FQs in the macrocycles is a fast exchange process that occurs on the NMR timescale.

### LEVO with SRsC1/βCD

In the presence of SRsC1 or βCD, the piperazine protons (H²_{LEVO} and H⁴_{LEVO}) of LEVO underwent significant upfield chemical shifts, suggesting these protons are involved in hydrophobic interactions with the interior of the host cavity. Without desiring to be bound by theory, the shift of H²_{LEVO} to higher magnetic fields could be attributed to magnetic anisotropy effects due to their location near the aromatic ring of SRsC1, which is rich in π-electrons. Other aromatic protons (H⁸_{LEVO} and H⁷_{LEVO}) showed progressive downfield shifts, mainly due to a variation in the polarity of their microenvironment when LEVO is inside the host cavity of SRsC1 and βCD. These changes indicated a shielding effect due to the interactions between guest and host, particularly hydrogen bonding and electrostatic interactions. Moreover, the downfield shift of H^{b}_{SRsC}1 of SRsC1 was attributed to a deshielding effect caused by π-H bonding with the piperazine moiety of LEVO. Interestingly, no proton shifts were observed for βCD in presence of LEVO.

### SRsC1 with CIPRO/NOR

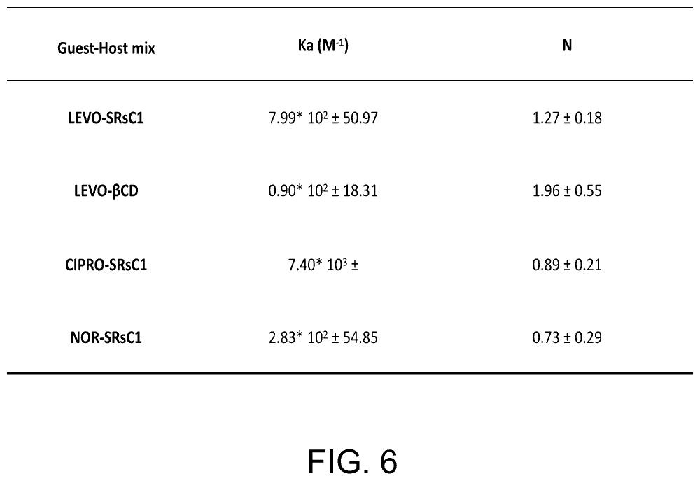

SRsC1 aromatic proton (H^{b}_{SRsC}1) shifted significantly downfield in the presence of structurally related guest molecules NOR and CIPRO. The aromatic protons (H⁵_{NOR}, H⁷_{NOR}, H⁶_{CIPRO} and H⁷_{CIPRO}) of NOR and CIPRO also shifted downfield in the NMR spectrum. These strongly deshielded effects were due to π-π bonding between host and guest. In contrast, no shift was observed for piperazine protons of NOR and CIPRO, suggesting that the aromatic ring system of the guest plays an important role in the inclusion process. Further, a strong upfield shift of bridging proton H^{d}_{sRsC1} in presence of NOR indicates shielding by methyl protons of NOR (H¹⁰_{NOR}). The titration data were also used to calculate the association constant and stoichiometry for the interaction of each FQ guest with the macrocycles. The Kₐ and N values of different host-guest mixtures are shown in FIG. 6.

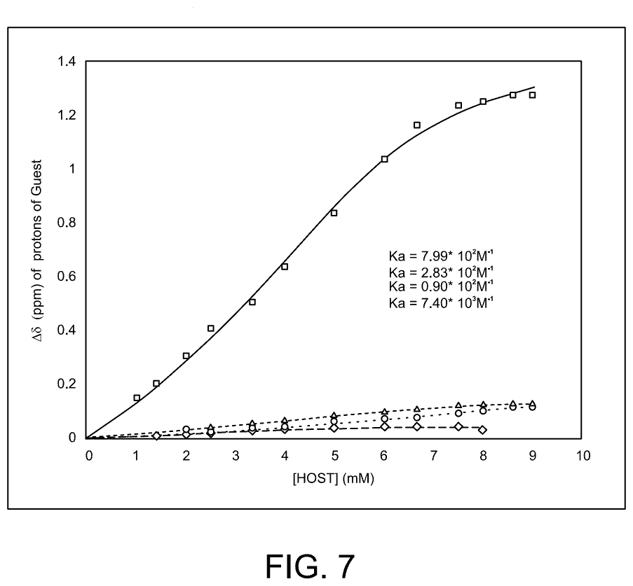

The variation of chemical shifts (Δδ) plotted as a function of host (SRsC1 and βCD) concentration for each FQ guest (LEVO, CIPRO, and NOR) are shown in FIG. 7. A global analysis was applied to determine the host/guest affinity. Comparing SRsC1 and βCD, it is confirmed that LEVO forms a tight complex with SRsC1. SRsC1 exhibited the strongest binding with CIPRO. The stoichiometry of the binding was observed to be 1:1, which corresponds to the continuous variation method, except for βCD and LEVO. While not desiring to be bound by theory, and as Jobs plots have some limitations for higher stoichiometry, it is believed that a 2:1 βCD-LEVO complex formation is likely. The obtained values of stability of the complexes are generally considered suitable for improvement of bioavailability and/or therapeutic efficacy of the guest.

### Intermolecular Interaction Between Host and FQ Guests

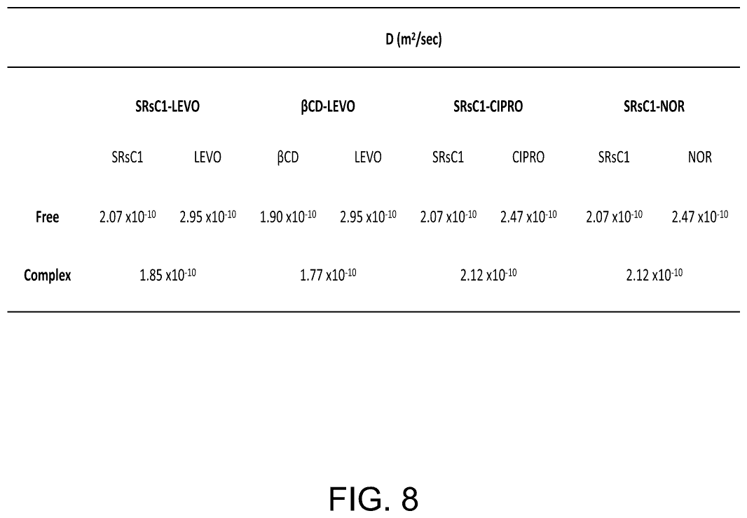

Diffusion-ordered spectroscopy (DOSY): The intrinsic diffusion coefficients (D) of molecules can be used to characterize intermolecular interactions in solution through DOSY. FIG. 8 is a table showing the D values of free host and guest molecules, as well as host-guest complexes at equimolar ratios. The FQs had higher D values than the host molecules, which is consistent with the FQs being smaller than the macrocycles. A molecule with slow diffusion is indicated by lower D values, implying that the molecule is heavier. The mixture of FQs with macrocycle diffuses more slowly than free components, indicating an increase in molecular size due to complexation. However, the diffusion coefficient D of SRsC1 was relatively unaffected by presence of structurally related guests (CIPRO and NOR). This is due to the small relative mass changes between free and complexed macrocycle. Meanwhile, the D values of both encapsulated CIPRO and NOR decreased in the presence of SRsC1, as shown in FIG. 8, providing evidence that these guests are included in the SRsC1 cavity and undergo slow diffusion. Similarly, the D value of LEVO decreased in the presence of SRsC1 and βCD, indicating that LEVO diffuses slowly because it is included in the cavities of both SRsC1 and βCD (see FIG. 8).

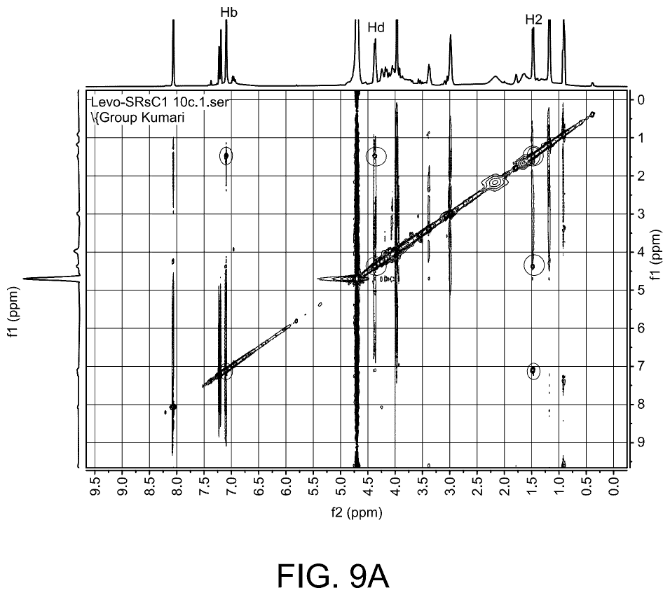

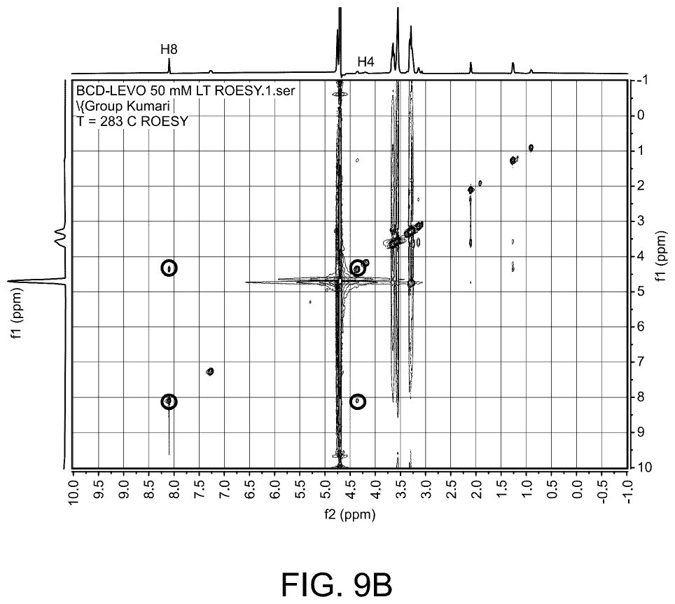

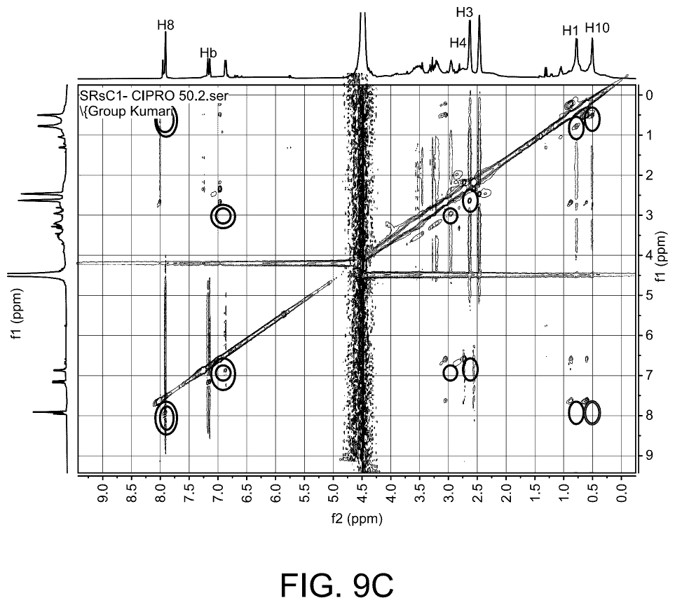

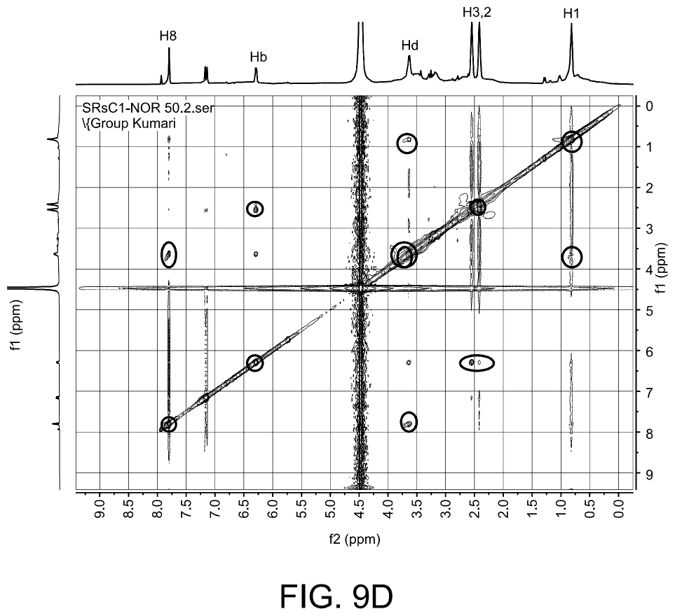

2D NOESY: The existence of cross correlation peaks in the NOE (nuclear Overhauser effect) between the protons of the guest and macrocycle signifies spatial connections and provides confirmation that the protons are located in close proximity to each other in space, at a distance of less than 4 angstroms. The full 2D NOE spectrum in FIG. 9A and FIG. 9B contains intermolecular cross-peaks between proton nuclei of FQs and SRsC1, whereas intramolecular cross peaks are observed with βCD. Strong intermolecular cross-peaks were present between the Hb and Hd protons of SRsC1 and the H2 proton of LEVO, although that with the bridging Hd proton of SRsC1 was weaker. These results indicate that the piperazine portion of LEVO enters the SRsC1 cavity while the aromatic and pyrido rings remain outside the cavity. In contrast, βCD show no observable intermolecular cross-peaks with LEVO protons, which suggests dimer formation of βCD and encapsulation of LEVO inside the dimer, in agreement with ¹H NMR results.

In the NOE spectra shown in FIG. 9C and FIG. 9D, both CIPRO and NOR guests exhibited two significant sets of intermolecular cross-peaks. These observations suggest that both guests interact in a similar manner with the SRsC1 host. Strong cross-peaks were observed between the piperazine protons (H2, H3, and H8 of NOR, and H3 and H4 of CIPRO) and the aromatic protons (Hb) of the host, indicating close interaction between these groups. Additionally, proximity was observed between the aliphatic protons (H1, H4, and H10 of CIPRO, and H1 of NOR) of the guests and the host protons (Hb and Hd). This observation is consistent with the results from the 1D proton NMR experiments, as no cross-peaks were observed for the aromatic protons of the guests. These results suggest the formation of specific interactions and complexation between SRsC1 and both CIPRO and NOR. The absence of cross-peaks for the aromatic protons of the guests in the NOE spectra may indicate a different mode of interaction or less direct involvement in complexation.

### Example 3. Solid-State Study of FQ Guests with Cycle Macromolecules Via FTIR

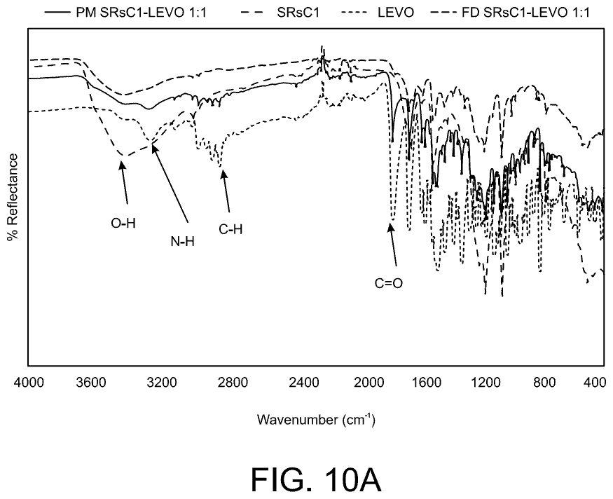

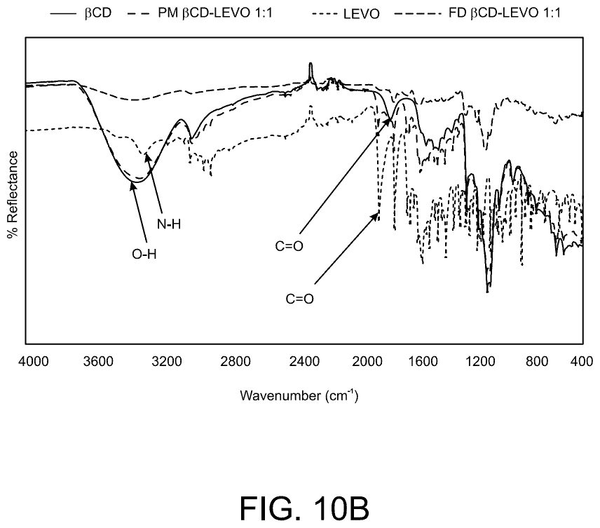

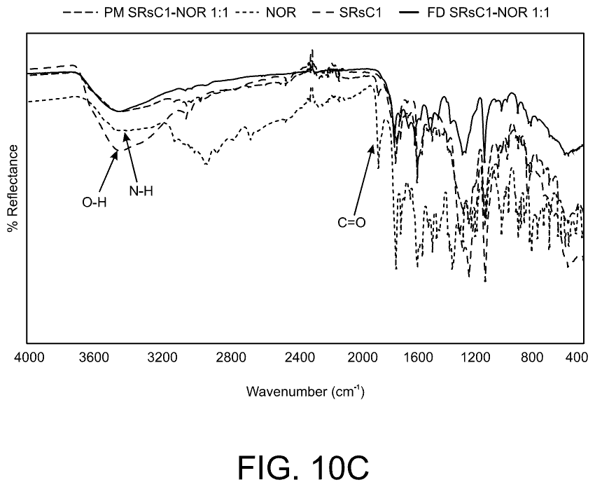

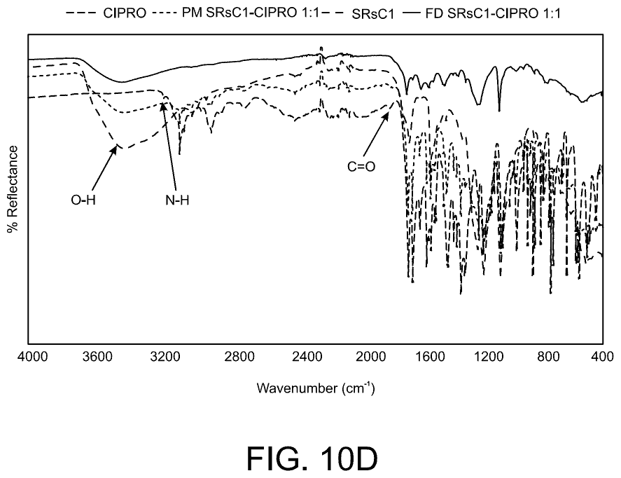

Variations in the infrared (IR) absorption peaks of different guest-host systems were analyzed to gain insights into their inclusion interactions. The FTIR spectra of SRsC1 and freeze-dried (FD) SRsC1-LEVO (1:1) were compared and a significant difference was observed (FIG. 10A). The disappearance of the peaks at 3233.6 cm⁻¹ and 2802.1 cm¹, corresponding to N—H and C—H stretching (aliphatic methyl group) of LEVO, respectively, indicates the formation of a host-guest complex. This disappearance can be attributed to the hydroxy (—OH) group of SRsC1 engaging in hydrogen bonding with the partially negative tertiary nitrogen of LEVO. Furthermore, the O—H stretching frequency of neat SRsC1 (3387.6 cm-1) red-shifted to 3406 cm⁻¹ after interacting with LEVO, suggesting a modification of hydrogen bonding in the FD SRsC1-LEVO complex. Additionally, the disappearance of the (C═O) peak of LEVO at 1719.5 cm-1 in FD SRsC1-LEVO mixture further confirms the involvement of the carbonyl group in complexation, consistent with the solution state study.

When LEVO forms a complex with βCD (FIG. 10B), the disappearance of the absorption peak at 3233.6 cm⁻¹, representing the N—H stretching in LEVO, indicates the formation of a complex. The βCD peak at 3274.6 cm⁻¹ (O—H stretching) decreased in intensity and shifted to a higher wavenumber, suggesting the formation of inclusion complex with LEVO. Additionally, the bands at 1642.7 cm⁻¹ and 1719.5 cm⁻¹ (C═O stretching) of both βCD and LEVO disappeared in the βCD-LEVO (1:1) complex, along with further reduction and masking observed in the fingerprint region. In the case of freeze-dried SRsC1-NOR (1:1), the peak corresponding to N—H stretching of the piperazinyl group in NOR was red-shifted to 3405.8 cm⁻¹ (FIG. 10C). This shift is likely a result of proton exchange via hydrogen bonding with SRsC1, leading to the overlap of the peak at 3339.1 cm⁻¹ in NOR with the peak at 3387.6 cm⁻¹ in SRsC1. A similar peak shift was observed in the freeze-dried complex of SRsC1-CIPRO in FIG. 10D. Variations in the shape, shift, and intensity of the IR absorption peaks of the guest or host molecules provide evidence of complex formation. Unlike those of the inclusion complexes, the FTIR spectra of the physical mixtures exhibited peaks corresponding to the individual hosts and FQs. Although the spectra of the physical mixtures appeared nearly identical to those of the host and FQ drugs alone, these results suggest that weak interactions occur in the physical mixtures.

### Example 4. Computational Analysis

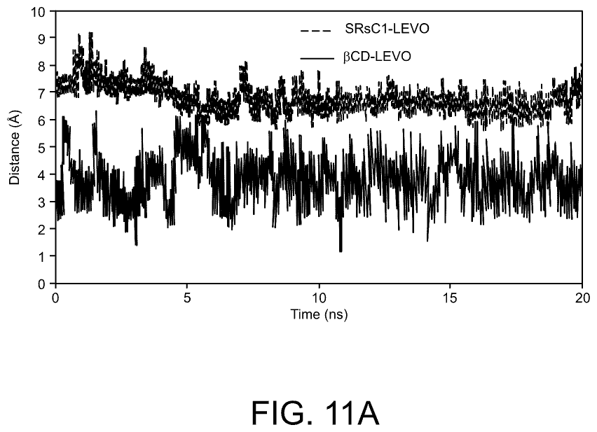

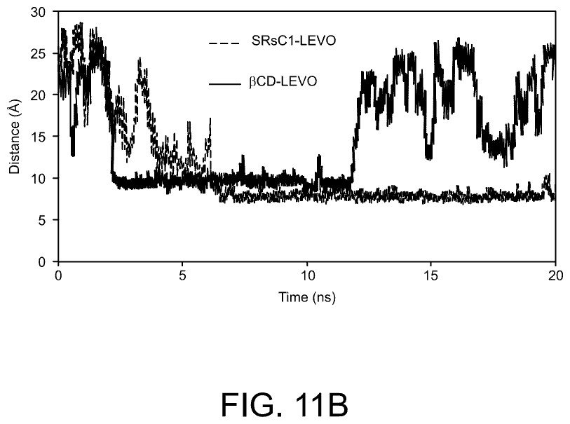

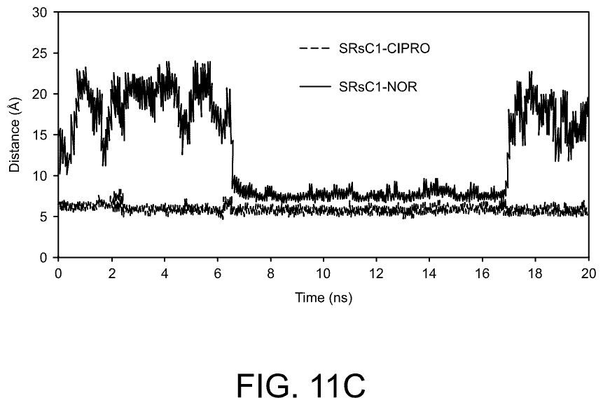

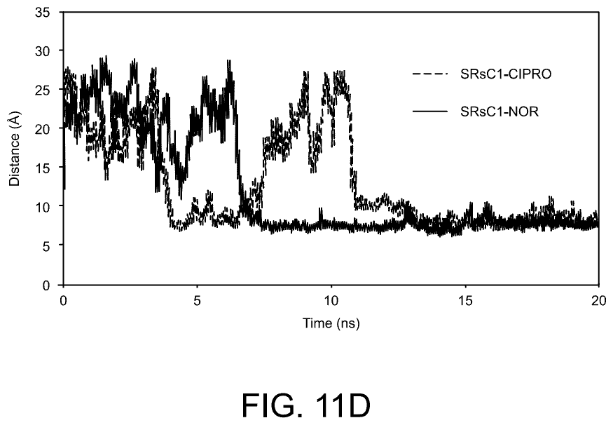

Classical MD simulations were utilized to probe possible interactions between SRsC1 and βCD host molecules with the FQ guest molecules. To better quantify the effect that the host-guest interaction has on the geometry of the host, lower-rim, upper-rim and mid radii of the hosts were monitored in addition to the center of mass (COM) distance between the host and guest. The interaction of the guests with the host has minimal effect on the SRsC1 or βCD framework, which maintain their original conformation; the sulfonato groups of SRsC1 do vary depending on whether the guest is interacting with the SRsC1. Production runs were carried out for the SRsC1 host with the piperazinyl group of the guest oriented within the cavity of SRsC1 and with the guest at least 10 Å above the host. When the piperazinyl group is oriented within the host cavity, the guest stays within the cavity of the host except for SRsC1-NOR, (FIG. 11A and FIG. 11C); although the sulfonato groups have relatively unhindered rotations, there tends to be an interaction between the sulfonato groups of SRsC1 and the guest. For this starting structure of SRsC1-NOR, a Na⁺ comes in proximity and interacts with SRsC1 during the NPT equilibration and disrupts the SRsC1-NOR interaction and leads to NOR interacting with the top rim of the SRsC1. For all host-guest combinations when the guest is initially oriented above the host, the guest moves closer and begins to interact with the side of the host or the upper-rim (FIG. 11B and FIG. 11D). Interestingly for this simulation of SRsC1-NOR, NOR starts to move toward the SRsC1 (around 4 ns of FIG. 11D) at the same time a Na⁺ is moving toward SRsC1; the Na⁺ interacts with 2-3 of the sulfonato groups from˜4-13 ns of this simulation, while NOR starts to interact with SRsC1 around 7 ns and as NOR starts to interact with the top rim of SRsC1, the Na⁺ is released.

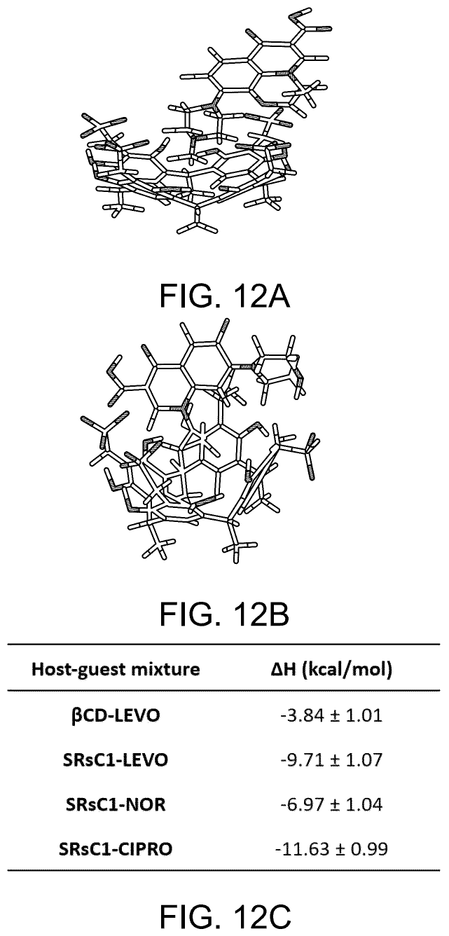

The additional set of production runs with the guest interacting with the cavity of the host for all host-guest combinations, with a constant number of solvent molecules, resulted in the guest interacting with the host throughout the entire production runs. The host-guest distance resembles that of FIG. 11A. The SRsC1 complexes interact more strongly due to the interaction of the sulfonato group with the guest. For LEVO (FIG. 12A) and NOR, the piperazine group stays within the cavity for the entire simulation; however, the most favorable binding enthalpy is observed for SRsC1-CIPRO (FIG. 12B, FIG. 12C). The cyclopropyl group of CIPRO stays within the SRsC1 cavity, which leads to possible hydrogen bonding interactions between the NH group of the CIPRO and the neighboring SO₃ group of the SRsC1 and the interaction of the SO₃ groups with the COOH end of CIPRO. These trends in binding enthalpies agree with the experimentally obtained Ka values.

### Example 5. Antibacterial Activity Comparison Between FQ Complexes Against Gram-Negative and Gram-Positive Bacteria

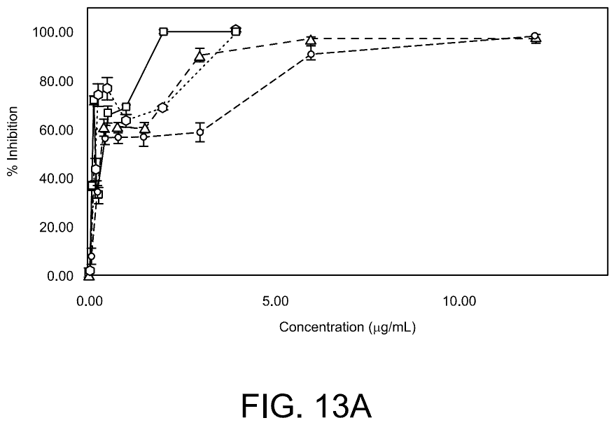

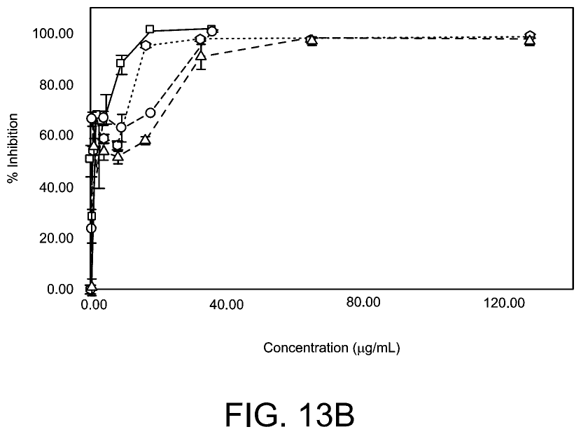

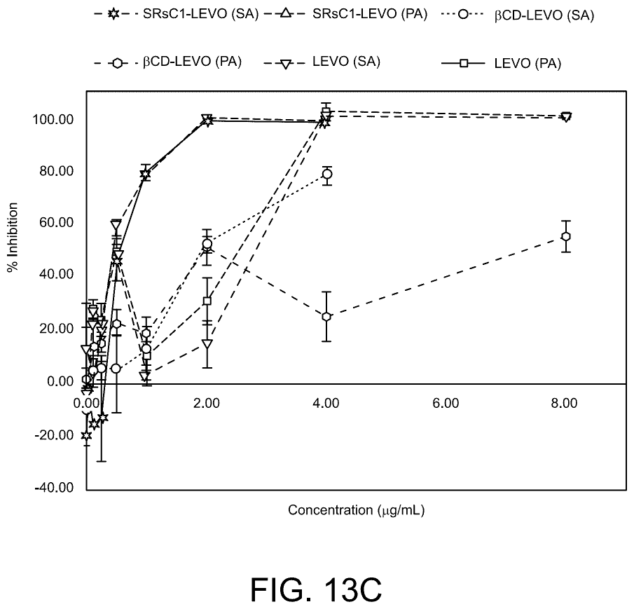

The antibacterial effectiveness of complexed FQs was assessed in comparison to uncomplexed FQs against Gram-positive Staphylococcus aureus (S. aureus) and Gram-negative Pseudomonas aeruginosa (P. aeruginosa) through a minimum inhibitory concentration (MIC) assay conducted at various concentrations. This involved serial dilution starting from 2× MIC on 96-well plates. The percentage growth inhibition of bacterial cells treated with complexed and uncomplexed FQs was determined by comparing with the untreated control (FIGS. 13A-13C). The concentrations tested for P. aeruginosa and S. aureus varied based on the MIC of individual antibiotics. Notably, the FQs exhibited a growth inhibitory effect on P. aeruginosa at 2- or 3-times lower concentrations compared to S. aureus.

All three tested molecules (FQs CIPRO, NOR, LEVO) and complexed FQs with the polyphenolic macrocycle (SRsC1) demonstrated a 90% inhibition of bacterial cells at higher concentration. However, this inhibitory effect decreased at lower concentrations against both bacterial strains. The percent inhibition curves for FQs and macrocycle complexed FQs exhibited a similar pattern, differing only at specific points where complexed FQs displayed a slightly lower inhibition for certain concentrations against both tested bacteria. As seen from MIC assay of S. aureus and P. aeruginosa in FIG. 13A, exposure of 12 μg/mL of both SRsC1-CIPRO and CIPRO caused 98.30±0.26% killing of S. aureus, compared to 4 μg/mL required for killing 100.67±0.39% P. aeruginosa bacteria. Similarly, the complex SRsC1-NOR and NOR alone demonstrated an inhibitory effect of 97.90±0.38% on S. aureus Cells at 128 μg/mL and 100.43±0.17% at 40 g/mL on P. aeruginosa cells (FIG. 13B). In comparing LEVO with SRsC1, we also evaluated LEVO complexed with βCD in FIG. 13C to assess the impact of the macrocycle on biological activity. LEVO complexed with βCD exhibited a 78.08±3.66% inhibitory effect on S. aureus cell growth and a 54.84±6.02% inhibitory effect on P. aeruginosa cell growth at 4 and 8 g/mL, respectively. This represents an approximately two-fold decrease compared to SRsC1-LEVO, as illustrated in FIG. 13C.

Although the complexed forms of NOR, CIPRO, and LEVO did not exhibit significant improvement compared to their free forms, they were established in a 1:1 stoichiometry with their respective host entities. Consequently, the measured quantity of antibiotic within the complex was reduced by half, compared to the standalone antibiotic doses administered for testing. This deliberate configuration ensured that, post-complexation, the efficacy of all three FQs (CIPRO, NOR and LEVO) remained consistently robust in presence of SRsC1. The sustained antimicrobial effectiveness at a diminished quantity underscores the unexpected efficacy of the 1:1 stoichiometric complexation strategy.

### Example 6. Effect of FQ Complexes Against Sensitive and Resistant Strains of P. Aeruginosa

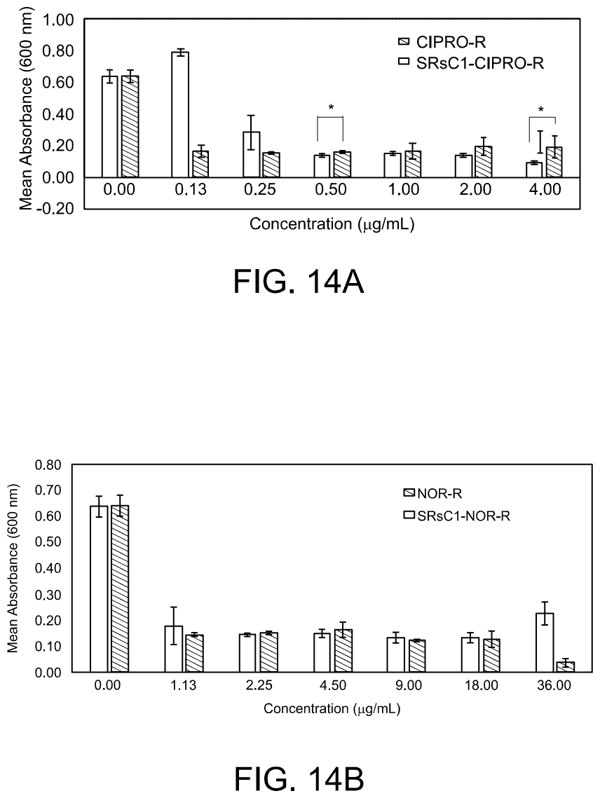

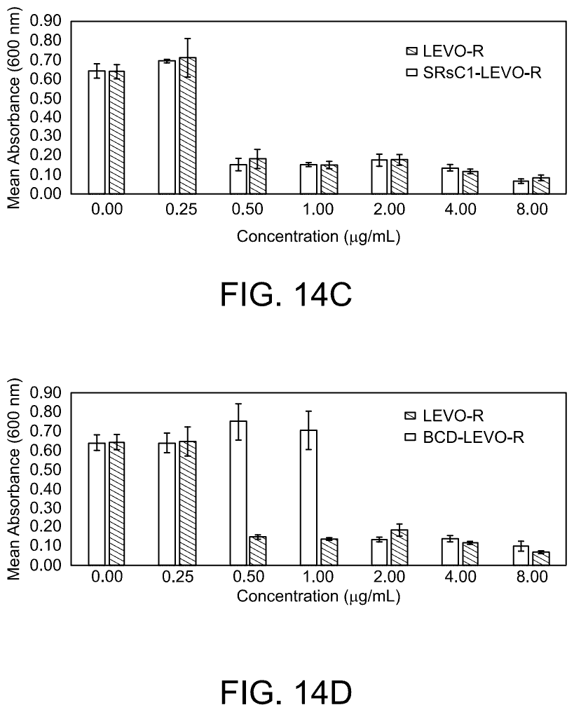

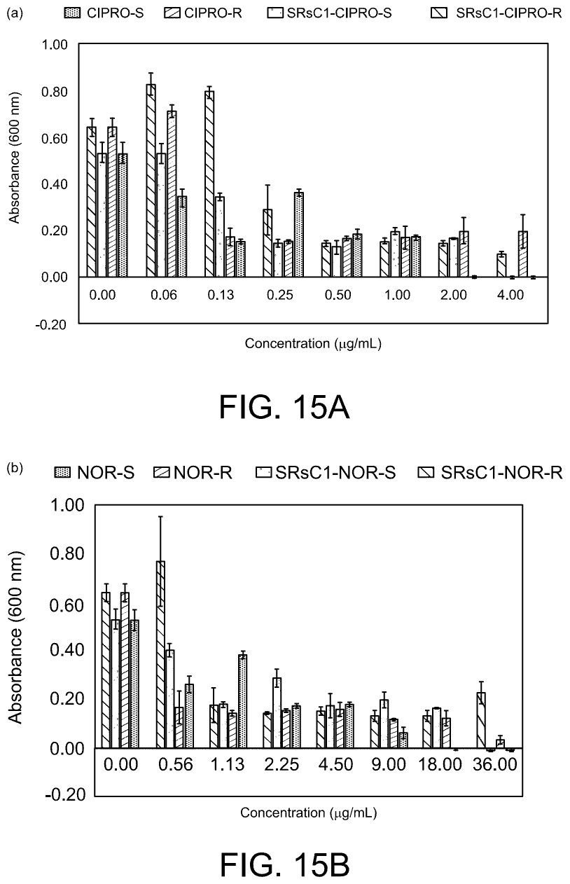

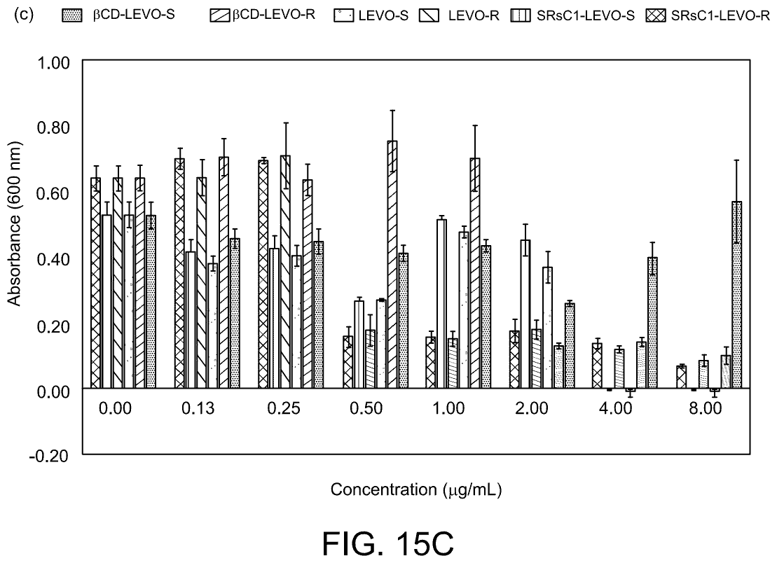

The inhibitory activity of the FQ complexes was compared with the free forms of the antibiotics against sensitive (S) and resistant (R) strains of P. aeruginosa. It was observed that both FQ complexes and free FQs exhibited lower inhibitory activity against resistant strains. However, a 2-fold increase in inhibitory effect was noted for SRsC1-CIPRO (0.09±0.01 absorbance @600 nm) compared to the host-free CIPRO (0.19±0.07 absorbance @600 nm) at 4 μg/mL, as shown in FIG. 15A. The inhibitory effects of SRsC1-CIPRO in FIG. 14A were observed to be significant at 4 and 0.5 μg/mL. The enhanced activity observed in SRsC1 is likely attributed to the orientation of CIPRO within the macrocycle. In this configuration, the piperazine moiety resides outside the macrocycle cavity, potentially facilitating bacterial outer membrane crossing through electrostatic interactions with the anionic groups of resistant bacteria outer membrane. This mechanism may not solely involve porin channels, suggesting a broader pathway for antibiotic uptake. On the other hand, NOR, a slightly different side chain counterpart of CIPRO, whether in its free form or complexed, maintained inhibitory effects against both strains (FIG. 15B). Although the complexed NOR was observed to be effective at lower concentration (2.25 and 4.50 μg/mL) against resistant strain of P. aeruginosa in FIG. 14B, the observed impact was deemed non-significant. When comparing two different macrocycles, SRsC1 and βCD, for LEVO in FIG. 15C, it was observed that complexes with polyphenolic macrocycles performed better against both sensitive and resistant strains. In contrast, βCD-LEVO exhibited greater inhibition towards resistant strains than sensitive strains at higher concentrations (8-2 μg/mL), but this was not comparable to LEVO itself. Moreover, the activity of SRsC1-LEVO and free LEVO remained consistent throughout the entire range of tested concentrations against resistant bacteria (FIG. 15C). Despite the inherent resistance of these strains, the SRsC1-LEVO complex exhibited a noteworthy inhibitory effect compared to βCD-LEVO in killing bacteria as seen in FIGS. 14C and 14D. At certain concentrations visible in FIGS. 14A-14D, it is evident that complexed FQs with SRsC1 are more effective against resistant bacteria than free FQs against sensitive strains. The concentrations showing comparable inhibitory effects by reducing the growth of resistant bacteria are predominantly at lower concentrations (4-0.5 μg/mL, 2.25-4.5 μg/mL, and 0.5-2.0 μg/mL) for SRsC1-complexed CIPRO, NOR, and LEVO compared to individual FQs. However, at sub-inhibitory concentrations, an increase in concentration is not always followed by an increase in the growth inhibition consistently against sensitive strains. While not desiring to be bound by theory, it is believed that this effect occurs due to the sub-inhibitory effects of an antibacterial agent that does not completely inhibit bacterial growth. Sub-inhibitory effects occur when bacteria are exposed to concentrations of an antibacterial agent below the MIC or alteration due to gene expression or adaptive responses.

### Example 7. Partition Coefficient of Complexed Fluoroquinolones

The experimental partition coefficient between octanol and phosphate buffer pH 7.4 (Ko/w) expressed in log P of free FQs and its complex with macrocyclic hosts was determined. The experimental log P values obtained for free FQs indicate they are hydrophilic in nature, which is approximately close to the reported values. LEVO observed to be least hydrophilic (log P=−0.41), followed by CIPRO and NOR (log P=−0.99 and −2.07). This is due to the zwitterionic nature of the FQs at the physiological pH, which is close to the isoelectric point (pH 7.0). It is presumed that the partitioning of quinolones was generally consistent with the knowledge that the neutral species of like compounds tends to be more lipophilic than the zwitterionic species. Unsurprisingly, despite having aromatic groups, polyphenolic macrocycle SRsC1 resulted in negative log P (−0.99), which indicates partitioning in aqueous phase, the same as CIPRO. Furthermore, macrocyclic complexes of FQs (SRsC1-LEVO, log P=0.34; SRsC1-NOR, log P=0.51 and SRsC1-CIPRO, log P=0.40) were found to be less hydrophilic compared to free FQs. The increased partition coefficient value may be the result of an increase in hydrophobicity of FQs after complexation with SRsC1. Similarly, βCD slightly decreases the hydrophilicity of LEVO by increasing the log P from −0.41 to −0.02.

Aspects of the present disclosure can be described with reference to the following numbered clauses, with preferred features laid out in dependent clauses.

1. A supramolecular complex comprising:
a macrocycle compound according to Formula I

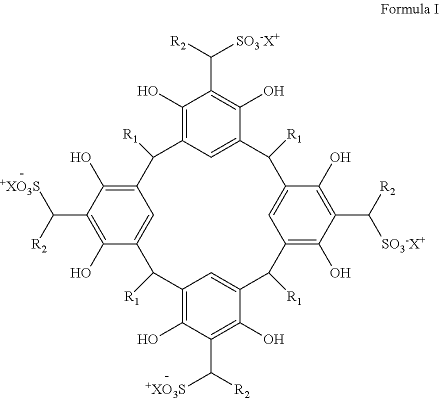

wherein:
R₁ is selected from C₁-C₂₀ alkyl, C₁-C₂₀ alkoxyl, and aryl; and
R₂ is selected from H, C₁-C₂₀ alkyl, C₁-C₂₀ alkoxyl, aryl, and (CH₂)ₙSO₃⁻X⁺, wherein n is an integer from 1 to 17 and X⁺ is a cation; and a fluoroquinolone compound.

2. The supramolecular complex according to claim 1, wherein X⁺ is a cation selected from the group consisting of Na⁺, K⁺, NH₄+, and C₅H₅NH⁺.

3. The supramolecular complex according to any of the preceding clauses, wherein the fluoroquinolone compound is selected from the group consisting of ciprofloxacin, levofloxacin, norfloxacin, delafloxacin, gemifloxacin, moxifloxacin, sparfloxacin, and ofloxacin.

4. The supramolecular complex according to any of the preceding clauses, wherein the fluoroquinolone compound is ciprofloxacin, levofloxacin, or norfloxacin.

5. The supramolecular complex according any of the preceding clauses, wherein the macrocycle compound is sulfonato-C-methylresorcin[4]arene (SRsC1).

6. The supramolecular complex according to any of the preceding clauses, comprising a binding stoichiometry of macrocycle to fluoroquinolone compound of 1:1.

7. A pharmaceutical composition comprising:
the supramolecular complex according to any of the preceding clauses or clauses 23-24; and a pharmaceutically-acceptable carrier.

8. The pharmaceutical composition according to clause 7, wherein the fluoroquinolone compound is selected from the group consisting of ciprofloxacin, levofloxacin, norfloxacin, delafloxacin, gemifloxacin, moxifloxacin, sparfloxacin, and ofloxacin.

9. The pharmaceutical composition according to any of clauses 7 or 8, wherein the composition is formulated for enteral, parenteral, topical, or ophthalmic administration.

10. A method of treating a bacterial infection in a subject in need thereof, the method comprising administering to the subject an effective amount of the supramolecular complex according to any of clauses 1-6 or 23-24.

11. The method according to clause 10, wherein the fluoroquinolone compound is selected from the group consisting of ciprofloxacin, levofloxacin, norfloxacin, delafloxacin, gemifloxacin, moxifloxacin, sparfloxacin, and ofloxacin.

12. The method according to any of clauses 10 or 11, wherein the bacterial infection is selected from the group consisting of a respiratory infection, a urinary tract infection, a joint infection, a bone infection, an intraabdominal infection, a soft tissue infection, a skin infection, an eye infection, a gastrointestinal infection, septicemia, typhoid fever, and anthrax.

13. The method according to any of clauses 10-12, wherein the bacterial infection is caused by bacteria selected from the group consisting of Acinetobacter spp., Actinomycetes spp., Bacillus spp., Bacteroides spp., Bordetella spp., Borrelia spp., Branhamella spp., Brucella spp., Burkholderia spp., Cedecea spp., Chlamydia spp., Citrobacter spp., Clostridium spp., Coxiella spp., Edwardsiella spp., Enterobacter spp., Enterobacteriaceae spp., Enterococcus spp., Escherichia spp., Erysipelothrix spp., Ewingella spp., Francisella spp., Fusospirocheta spp., Gardenerella spp., Haemophilus spp., Hafnia spp., Klebsiella spp., Kluyvera spp., Legionella spp., Listeria spp., Micrococcus spp., Morganella spp., Mycobacteria spp., Mycoplasma spp, Neisseria spp., Nocardia spp., Pasteurella spp., Peptostreptococcus spp., Proteus spp., Providencia spp., Pseudomonas spp., Rahnella spp., Rickettsia spp., Salmonella spp., Serratia spp., Shigella spp., Spirillum spp., Spirochaeta spp., Staphylococcus spp., Streptobacillus spp., Streptococcus spp., Streptomyces spp., Tatumella spp., Treponema spp., Trichomonas spp., Ureaplasma spp., Vaginalis spp., Vibrio spp., Xanthomonas spp., and Yersinia spp.

14. The method according to any of clauses 10-13, wherein the bacterial infection is caused by Gram-positive or Gram-negative bacteria.

15. The method according to any of clauses 10-14, wherein risk of developing antibiotic resistance to the fluoroquinolone compound is reduced.

16. The method according to any of clauses 10-15, wherein the bacteria are resistant to the fluoroquinolone.

17. A method of killing or inhibiting the growth of bacteria, the method comprising contacting the bacteria with an effective amount of the supramolecular complex according to any of clauses 1-6 or 23-24.

18. The method according to clause 17, wherein the fluoroquinolone compound is selected from the group consisting of ciprofloxacin, levofloxacin, norfloxacin, delafloxacin, gemifloxacin, moxifloxacin, sparfloxacin, and ofloxacin.

19. The method according to any of clauses 17-18, wherein the bacteria are selected from the group consisting of Acinetobacter spp., Actinomycetes spp., Bacillus spp., Bacteroides spp., Bordetella spp., Borrelia spp., Branhamella spp., Brucella spp., Burkholderia spp., Cedecea spp., Chlamydia spp., Citrobacter spp., Clostridium spp., Coxiella spp., Edwardsiella spp., Enterobacter spp., Enterobacteriaceae spp., Enterococcus spp., Erwinia spp., Escherichia spp., Erysipelothrix spp., Ewingella spp., Francisella spp., Fusospirocheta spp., Gardnerella spp., Haemophilus spp., Hafnia spp., Klebsiella spp., Kluyvera spp., Legionella spp., Listeria spp., Micrococcus spp., Morganella spp., Mycobacteria spp., Mycoplasma spp., Neisseria spp., Nocardia spp., Pasteurella spp., Peptostreptococcus spp., Proteus spp., Providencia spp., Pseudomonas spp., Rahnella spp., Rickettsia spp., Salmonella spp., Serratia spp., Shigella spp., Spirillum spp., Spirochaeta spp., Staphylococcus spp., Streptobacillus spp., Streptococcus spp., Streptomyces spp., Tatumella spp., Treponema spp., Trichomonas spp., Ureaplasma spp., Vaginalis spp., Vibrio spp., Xanthomonas spp., and Yersinia spp.

20, The method according to any of clause 17-19, wherein the bacteria are Gram-positive or Gran-negative.

21. A method of disrupting a bacterial biofilm, the method comprising contacting the bacterial biofilm with an effective amount of the supramolecular complex according to any of clauses 1-6 or 23-24.

22. The method according to clause 21, wherein the fluoroquinolone compound is selected from the group consisting of ciprofloxacin, levofloxacin, norfloxacin, delafloxacin, gemifloxacin, moxifloxacin, sparfloxacin, and ofloxacin.

23. A supramolecular complex comprising:
sulfonato-C-methylresorcin[4]arene (SRsC1); and a fluoroquinolone compound;
or a salt thereof.

24. The supramolecular complex according to clause 23, wherein the fluoroquinolone compound is selected from the group consisting of ciprofloxacin, levofloxacin, norfloxacin, delafloxacin, gemifloxacin, moxifloxacin, sparfloxacin, and ofloxacin.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” is used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something less than exact.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A supramolecular complex comprising:
a macrocycle compound according to Formula I 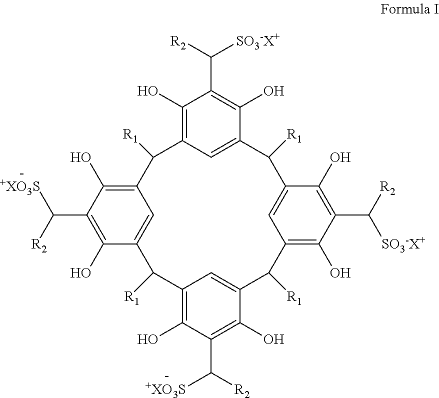
wherein:
R₁ is selected from C₁-C₂₀ alkyl, C₁-C₂₀ alkoxyl, and aryl; and
R₂ is selected from H, C₁-C₂₀ alkyl, C₁-C₂₀ alkoxyl, aryl, and (CH₂)ₙSO₃⁻X⁺, wherein n is an integer from 1 to 17, and wherein X⁺ is a cation; and
a fluoroquinolone compound.

2. The supramolecular complex according to claim 1, wherein X⁺ is selected from the group consisting of Na⁺, K⁺, NH₄+, and C₅H₅NH⁺.

3. The supramolecular complex according to claim 1, wherein the fluoroquinolone compound is selected from the group consisting of ciprofloxacin, levofloxacin, norfloxacin, delafloxacin, gemifloxacin, moxifloxacin, sparfloxacin, and ofloxacin.

4. The supramolecular complex according to claim 3, wherein the fluoroquinolone compound is ciprofloxacin, levofloxacin, or norfloxacin.

5. The supramolecular complex according to claim 1, wherein the macrocycle compound is sulfonato-C-methylresorcin[4]arene (SRsC1).

6. The supramolecular complex according to claim 1, comprising a binding stoichiometry of macrocycle to fluoroquinolone compound of 1:1.

7. A pharmaceutical composition comprising:
the supramolecular complex according to claim 1; and
a pharmaceutically-acceptable carrier.

8. The pharmaceutical composition according to claim 7, wherein the fluoroquinolone compound is selected from the group consisting of ciprofloxacin, levofloxacin, norfloxacin, delafloxacin, gemifloxacin, moxifloxacin, sparfloxacin, and ofloxacin.

9. The pharmaceutical composition according to claim 7, wherein the composition is formulated for enteral, parenteral, topical, or ophthalmic administration.

10. A method of treating a bacterial infection in a subject in need thereof, the method comprising administering to the subject an effective amount of the supramolecular complex according to claim 1.

11. The method according to claim 10, wherein the fluoroquinolone compound is selected from the group consisting of ciprofloxacin, levofloxacin, norfloxacin, delafloxacin, gemifloxacin, moxifloxacin, sparfloxacin, and ofloxacin.

12. The method according to claim 10, wherein the bacterial infection is selected from the group consisting of a respiratory infection, a urinary tract infection, a joint infection, a bone infection, an intraabdominal infection, a soft tissue infection, a skin infection, an eye infection, a gastrointestinal infection, septicemia, typhoid fever, and anthrax.

13. The method according to claim 12, wherein the bacterial infection is caused by bacteria selected from the group consisting of Acinetobacter spp., Actinomyces spp., Bacillus spp., Bacteroides spp., Bordetella spp., Borrelia spp., Branhamella spp., Brucella spp., Burkholderia spp., Cedecea spp., Chlamydia spp., Citrobacter spp., Clostridium spp., Coxiella spp., Edwardsiella spp., Enterobacier spp., Enterobacteriaceae spp., Enterococcus spp., Escherichia spp., Erysipelothrix spp., Ewingella spp., Francisella spp., Fusospirocheta spp., Gardenerella spp., Haemophilus spp., Hafnia spp., Klebsiella spp., Kluyvera spp., Legionella spp., Listeria spp., Micrococcus spp., Morganella spp., Mycobacteria spp., Mycoplasma spp., Neisseria spp., Nocardia spp., Pasteurella spp., Peptostreptococcus spp., Proteus spp., Providencia spp., Pseudomonas spp., Rahnella spp., Rickettsia spp., Salmonella spp., Serratia spp., Shigella spp., Spirillum spp., Spirochaeta spp., Staphylococcus spp., Streptobacillus spp., Streptococcus spp., Streptomyces spp., Tatumella spp., Treponema spp., Trichomonas spp., Ureaplasma spp., Vaginalis spp., Vibrio spp., Xanthomonas spp., and Yersinia spp.

14. The method according to claim 12, wherein the bacterial infection is caused by Gram-positive or Gram-negative bacteria.

15. The method according to claim 10, wherein risk of developing antibiotic resistance to the fluoroquinolone compound is reduced.

16. The method according to claim 10, wherein the bacteria are resistant to the fluoroquinolone.

17. A method of killing or inhibiting the growth of bacteria, the method comprising contacting the bacteria with an effective amount of the supramolecular complex according to claim 1.

18. The method according to claim 17, wherein the fluoroquinolone compound is selected from the group consisting of ciprofloxacin, levofloxacin, norfloxacin, delafloxacin, gemifloxacin, moxifloxacin, sparfloxacin, and ofloxacin.

19. The method according to claim 17, wherein the bacteria are selected from the group consisting of Acinetobacter spp., Actinomycetes spp., Bacillus spp., Bacteroides spp., Bordetella spp., Borrelia spp., Branhamella spp., Brucella spp., Burkholderia spp., Cedecea spp., Chlamydia spp., Citrobacter spp., Clostridium spp., Coxiella spp., Edwardsiella spp., Enterobacter spp., Enterobacteriaceae spp., Enterococcus spp., Erwinia spp., Escherichia spp., Erysipelothrix spp., Ewingella spp., Francisella spp., Fusospirocheta spp., Gardenerella spp., Haemophilus spp., Hafnia spp., Klebsiella spp., Kluyvera spp., Legionella spp., Listeria spp., Micrococcus spp., Morganella spp., Mycobacteria spp., Mycoplasma spp., Neisseria spp., Nocardia spp., Pasteurella spp., Peptostreptococcus spp., Proteus spp., Providencia spp. Pseudomonas spp., Rahnella spp., Rickettsia spp., Salmonella spp., Serratia spp., Shigella spp., Spirillum spp., Spirochaetes spp., Staphylococcus spp., Streptobacillus spp., Streptococcus spp., Streptomyces spp., Tatumella spp., Treponema spp., Trichomonas spp., Ureaplasma spp., Vaginalis spp., Vibrio spp., Xanthomonas spp., and Yersinia spp.

20. The method according to claim 17, wherein the bacteria are Gram-positive or Gram-negative.

21. A method of disrupting a bacterial biofilm, the method comprising contacting the bacterial biofilm with an effective amount of the supramolecular complex according to claim 1.

22. The method according to claim 21, wherein the fluoroquinolone compound is selected from the group consisting of ciprofloxacin, levofloxacin, norfloxacin, delafloxacin, gemifloxacin, moxifloxacin, sparfloxacin, and ofloxacin.

23. A supramolecular complex comprising:
sulfonato-C-methylresorcin[4]arene (SRsC1); and
a fluoroquinolone compound;
or a salt thereof.

24. The supramolecular complex according to claim 23, wherein the fluoroquinolone compound is selected from the group consisting of ciprofloxacin, levofloxacin, norfloxacin, delafloxacin, gemifloxacin, moxifloxacin, sparfloxacin, and ofloxacin.
